(19) United States
(12) Patent Application Publication
(10) Pub. No.: US 2022/0028483 A1
(43) Pub. Date: Jan. 27, 2022

(54) SYSTEMS AND METHODS FOR CLASSIFYING TUMORS

(71) Applicant: PRESIDENT AND FELLOWS OF HARVARD COLLEGE, Cambridge, MA (US)

(72) Inventors: Doga C. GULHAN, Cambridge, MA (US); Peter J. PARK, Cambridge, MA (US)

(73) Assignee: PRESIDENT AND FELLOWS OF HARVARD COLLEGE, Cambridge, MA (US)

(21) Appl. No.: 17/277,647

(22) PCT Filed: Sep. 18, 2019
(86) PCT No.: PCT/US2019/051663

§ 371 Date: Mar. 18, 2021

Related U.S. Application Data
(60) Provisional application No. 62/735,674, filed on Sep. 24, 2018.

(51) Int. Cl. G16B 20/20; G16B 40/00; G16H 50/70; G16H 10/40; G06N 20/20

(52) U.S. Cl. CPC G16B 20/20; G16B 40/00; G06N 20/20; G16H 10/40; G16H 50/70

(57) ABSTRACT

Disclosed are systems and methods that can identify mutational signatures relevant to various cancers and/or treatments using genetic data from the tumors. This includes using a likelihood based measure, to compare clusters of tumor spectrums when the sample has sequenced only a sub-set of the genes with a targeted panel. In one example, by enabling panel-based identification of mutational signatures, our method substantially increases the number of patients that may be considered for treatments targeting HR deficiency.

### CROSS-REFERENCE TO RELATED APPLICATION

This Application claims benefit under 35 U.S.C. § 119(e) of the U.S. Provisional Application No. 62/735,674 filed Sep. 24, 2018, the contents of which are incorporated herein by reference in their entirety.

### FIELD OF THE INVENTION

The present invention is directed to classification and treatment of tumors using genetic analysis.

### BACKGROUND

The following description includes information that may be useful in understanding the present invention. It is not an admission that any of the information provided herein is prior art or relevant to the presently claimed invention, or that any publication specifically or implicitly referenced is prior art.

Mutational signature analysis has emerged as a powerful approach for investigating the mutational processes that generate somatic mutations. Conceptually, this analysis is based on the observation that different mutational processes often generate specific base-pair changes, typically in particular nucleotide contexts (Nik-Zainal et al., 2012). For instance, ultraviolet radiation generally results in C-to-T changes, often with C flanked by a C or T on the 5′ side. In its popular form (Alexandrov et al., 2013b, 2013a), this analysis computes a vector of 96 triplets (6substitution subtypes, C>A, C>G, C>T, T>A, T>C, and T>G; each flanked by 4 types on the 5′ and 3′ sides) for a set of genomes and deconvolves the observed mutational spectra into independent components. Once such mutational ‘signatures’ are defined from a large collection of sequencing datasets, it is also possible to map the mutational spectra of a new sample to a combination of signatures from the pre-defined catalog.

Application of this concept on thousands of tumor exomes and whole-genomes (WGS) has led to a catalog of nearly forty mutational signatures operative in cancer (Alexandrov et al., 2013); recently, this catalog has been extended further (Alexandrov et al., 2018). There is no single pre-defined signature catalog; as more data are accumulated, researchers are generating improved catalogs. Some of these signatures have been matched to specific mutational processes, both endogenous (e.g., replication clock, APOBEC cytosine deaminases, defects of DNA repair machineries) and exogenous (e.g., smoking carcinogens, UV radiation), although the majority of signatures still remain uncharacterized. Several signatures were experimentally validated by inactivation of key molecules in cell lines/organoids that result in mutational patterns resembling the predicted signature (Burns et al., 2013; Drost et al., 2017; Fedeles et al., 2017; Haradhvala et al., 2018; Meier et al., 2018; Nik-Zainal et al., 2015; Ohno et al., 2014; Zou et al., 2018).

In breast cancer, a landmark study of 560 whole genomes (Nik-Zainal et al., 2016) and subsequent studies (Davies et al., 2017; Polak et al., 2017) revealed that one of these signatures—‘Signature 3’—corresponds to a defect in the homologous recombination (HR) machinery (see Supplementary FIG. 1). This signature is observed in tumors with complete inactivation of BRCA1/2. This inactivation can occur by germline and somatic point mutations, loss of heterozygosity (LOH) due to structural variations, hyper-methylation of BRCA1 promoters, or loss-of-function mutations of PALB2 and RAD51D (Polak et al., 2017). Experimentally, Signature 3 was observed in BRCA −/− isogenic cell lines, providing a direct evidence of its association with HR defect (Zámborszky et al., 2017).

### SUMMARY

Importantly, there is increasing evidence that Signature 3 is not limited to those with a germline mutation in BRCA1/2 or other known HR-related genes (Nik-Zainal et al., 2016; Northcott et al., 2017; Polak et al., 2017). This is clinically relevant because those without a mutation in a known HR gene but still having Signature 3 may benefit from treatments that target selective vulnerability of HR-defect cancers. A recent study using breast cancer organoids, for example, has shown that the high burden of Signature 3 mutations is associated with a better response to PARP (poly [ADP-ribose] polymerase) inhibitors (Sachs et al., 2018). Inhibitors of PARP enzymes cause multiple double-strand breaks, and tumor cells that cannot repair the breaks due to HR defect do not survive.

Accordingly, new systems and methods have been developed for detecting various mutational signatures from sequencing data of an individual, including signature 3. Although previous methods have addressed identification of HR defect through mutational signatures (Davies et al., 2017; Polak et al., 2017), they were limited to exome or whole-genome data, thus hampering its use in clinical practice. For the most common genetic testing platform in oncology clinics—targeted sequencing panels—the number of mutations identifiable is far too small for standard signature analysis. A recent panel-based study of >10,000 cancer patients, for example, could perform signature analysis for only 6% of the samples with the highest mutational burden (Zehir et al., 2017).

Described herein is a newly developed computational tool called SigMA (Signature Multivariate Analysis) that uses a likelihood-based approach to detect signatures including Signature 3 from low mutation counts. Thus, application of this method has the potential to vastly expand the number of samples that will benefit from treatments available for HR-defect tumors and other types of tumors.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 depicts an example of an overview of a system used to classify tumors;

FIG. 2 depicts a flow chart showing an example process for implementing a classifier according to the present disclosure;

FIGS. 3A-3E depict an example of an overview for Signature 3 prediction. FIG. 3A depicts a graph 731-breast cancer WGS samples grouped based on their fractional signature compositions. FIG. 3B depicts the same graphs as for 3A, but for other tumor types. FIG. 3C is a flow chart depicting key steps in one example of the disclosed analysis. To estimate sensitivity and false positive rate, the system utilized simulated exomes and panels generated by subsampling from WGS data. To generate the SigMA score for a new sample, several statistics were calculated and combined to determine the category to which the sample is likely to belong. For low SNV count cases, for instance, the likelihood model automatically receives more weight in the prediction. FIG. 3D depicts a graph showing a number of SNVs for WGS samples and the subsampled panels. There is a three orders-of-magnitude reduction in the number of SNVs for panels compared to WGS. The dashed horizontal line marks five mutations, the minimum we require for inference. FIG. 3E depicts graphs bar graphs showing the spectra and score in the simulated panel example. FIG. 3F depicts the confusion matrix, showing the fraction of samples predicted to be a given signature category with SigMA (x-axis) for each WGS signature groups (y-axi s).

FIGS. 4A-4D depict the performance of one example of the disclosed systems and methods. FIG. 4A depicts, for three sequencing platforms (WGS, exomes, and panel, where the last two is simulated from WGS), graphs showing distributions of four measures (cosine similarity, exposure, and likelihood, and SigMA score) for Signature 3-positive and negative tumors as determined by NMF analysis using WGS data. FIG. 4B depicts a graphs showing the sensitivity versus FPR for SigMA compared to stand-alone use of cosine similarity and NNLS exposure for panel simulations. FIG. 4C depicts a graph showing the higher sensitivity of SigMA to detect Signature 3 compared to cosine similarity and two NNLS-based tools for panels, exome, and WGS. Error bars denote the standard error. FPR was fixed at 10% for panels and at 5-8% for exome and WGS. FIG. 4D depicts graph showing increased sensitivity when Signature 3 exposure is high (0.88 for FPR 10% and 0.71 for FPR 1%). The samples are divided into high/low exposure groups based on the median exposure;

FIGS. 5A-5C illustrate an example of validation of SigMA on MSK-IMPACT data. FIG. 5A depicts a graph showing the total number of mutations in the panel data split according to the classification by SigMA. A large number of cases have 5-10 mutations; the number of mutations in each category is similar to that of simulated panels shown in FIG. 3D. FIG. 5B depicts graphs showing the average mutational spectra of tumors classified to be signature 3-positive or—negative by SigMA. The first two rows correspond to modest (10% FPR) and stringent (1% FPR) criteria. These spectra resemble those from the simulated panels (third row), which are grouped based on WGS data. The horizontal bars below each spectrum show the fractions of signatures found by decomposing the average spectra by NNLS. FIG. 5C depicts graphs showing the CN balance for WGS samples with and without Signature 3 based on the NMF analysis. MSK panel samples split according to SigMA classification show similar differences in CN imbalance, as inferred from SNP array data for these samples;

FIGS. 6A-6B illustrate an example of experimental validation using drug response data. FIG. 6A depicts a graph showing IC50 (uM) values of olaparib in cell lines from different tumor types for Signature 3-positive and -negative tumors. Few cell lines, which fall above the maximum scale of the y-axis, are represented at a placeholder high IC50, while the actual values are written below them in parentheses. Next to the name of each tumor type the number of cell lines is shown. P-values are computed by the Kolmogorov-Smirnov test. FIG. 6B depicts a graph showing combined results for IC50 values for all tumor types, after normalization in each tumor type by subtracting the mean and dividing by the standard deviation. FIGS. 6C and 6D illustrate graphs showing the same as FIGS. 6A, B but for veliparib;

FIG. 7 depicts a table showing examples of how the disclosed systems and methods have been applied to various tumor types using simulated panels. The types include validation that these systems and methods may be used with respect to at least ovarian cancer, osteosarcoma, medulloblastoma, breast cancer, uterus corpus endometrial carcinoma, prostate adenocarcinoma, stomach adenocarcinoma, pancreas adenocarcinoma, pancreatic neuroendocrine cancer, oesophageal carcinoma, and Ewing's sarcoma; and

FIG. 8 depicts a flow chart showing an example process for implementing a classifier according to the present disclosure.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Definitions

As used herein, a “subject” means a cancer patient, a model organism most commonly rodents, or a model experiment such as a cancer cell line.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments as described herein, nucleic acid sequence data can be obtained in the format provided by different sequencing platforms that output raw genetic data. As a non-limiting example, nucleic acid sequence data can be provided in at least one of the following formats: raw sequence read format, plain sequence format, Federal Acquisition Streamlining Act-All (FASTA) format, FASTA Quality score (FASTQ) format, European Molecular Biology Laboratory (EMBL) format, binary base call (BCL) format, Variant Call Format (VCF), Binary Alignment Map (BAM) format, Sequence Alignment Map (SAM) format, Wisconsin GCG format, GCG-Rich Sequence Format (GCG-RSF), GenBank format, IG format, CRAM format, Standard Flowgram Format (SFF), Hierarchical Data Format (HDF; e.g., HDF4, HDF5), Color Space FASTA (CSFASTA) format, Sequence Read Format (SRF), Native Illumina format, or QSEQ format, Mutation Annotation Format (MAF).

### Overview

Disclosed are systems and methods that can identify mutational signatures relevant to various cancers and/or treatments using genetic data from the tumors. This includes using a likelihood-based measure to identify the relevant signature for the sample when only a sub-set of the genes has been sequenced with a targeted panel.

### System

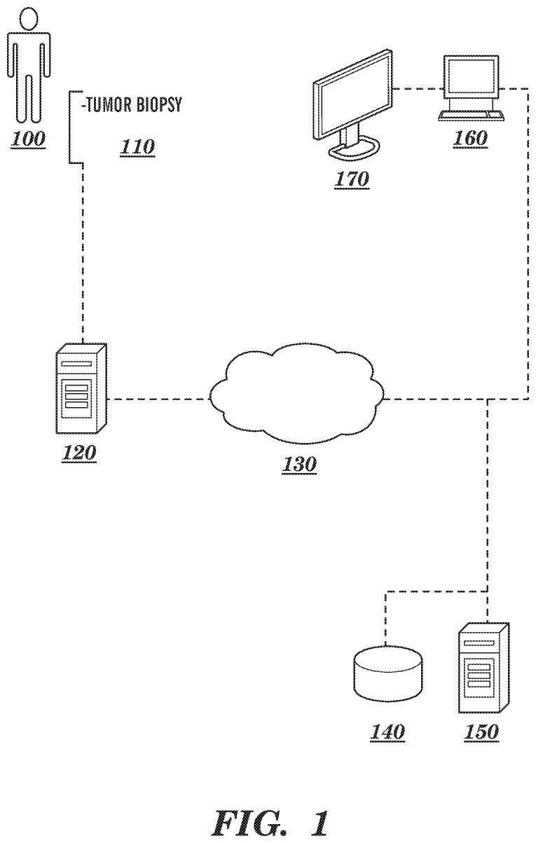

FIG. 1 illustrates an example overview of a system for implementing the current disclosure. The system may include a subject 100 and a variety of subject samples 110 that may include biopsies of various tumors.

Additionally, the system includes a gene sequencer 120 for processing the genetic information in samples from the subject. The gene sequencer 120 may be any suitable sequencer for determining the DNA sequences of the bacteria contained in the samples 110 from the subject 100 or the DNA of the biopsied or collected tissue. For instance, suitable gene sequencing systems may include the MiSeq, NextSeq, HiSeq, NovaSeq, Oxford Nanopore, and PacBio sequencers. However, additional sequencing technologies that are suitable may be utilized for instance RNA sequencing may also be used to identify somatic mutations on the DNA with less specificity.

The gene sequencer 120 may be connected to a network 130. Network 130 may be an internal network, external network, the internet or any other system or method for electronic communication. In other examples, the data may be manually removed from gene sequencer 120.

Network 130 may be connected to computing device 160 and display 170. Computing device 160 may be any suitable computing device 160, including a desktop computer, server (including remote servers), mobile device, or other suitable computing device 160. Additionally, network 130 may be connected to a server 150 and database 140. In some examples, algorithms, and other software may be stored in database 140 and run on server 150. Additionally, subject 100 data and other genetic information may be stored in database 140.

### Methods—Sequencing Samples

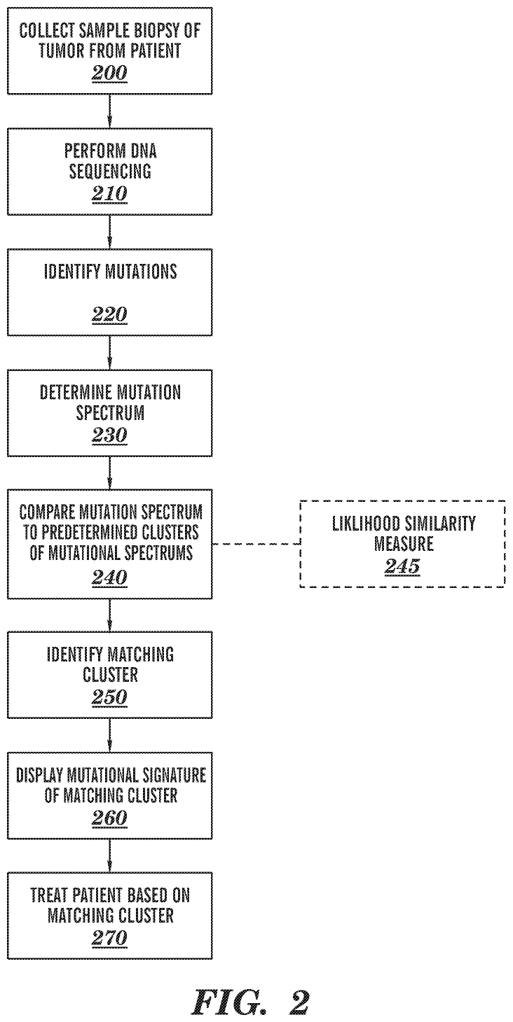

FIG. 2 illustrates an example of a method for classifying a subject's 100 sample 110 and treating a subject 100. For instance, first a biopsy sample 110 may be collected from a subject 200. This may be performed by a caregiver using any suitable methods.

Next, the DNA from the sample 110 may be sequenced 210 to output genetic data. For instance, prepared DNA may be processed with a high throughput sequencer 120, to output a FASTQ/FASTA file or other file containing raw genetic information.

The genes from the sample that may be sequenced may be a subset of genes or the entire genome. For instance, the subset of genes maybe 50-20,000 genes, 300-700 genes, at least 300 genes, or 410 genes. In some examples, a subset of the genes will be sequenced to perform a panel analysis of mutations in the subset of genes (or of the whole genome) to output a set of mutations for the sample. For instance, a variety of mutational panels could be utilized, for instance the MSK-IMPACT panel as described in the “Evaluation of Automatic Class III Designation for MSK-IMPACT, available at https://wwww.accessdata.fda.ov/cdrh docs/reviews/DEN170058.pdf, the content of which is incorporated by reference in its entirety. Accordingly, the result of this process will be the output of a set of somatic mutations based on the subset of sequenced genes or the whole genome.

Then, the sequence data may be transmitted over a network 130 to be stored in a database 140 by a server 150, or further processed on local memory. In some examples, the server 150 may then perform further processing on the sequence data or sequence data files.

### Methods—Mutation Spectrum Analysis

Next, the system may process the set of somatic mutations to output a sample mutation spectrum 230. The mutational spectrum may be vector, table, list or other compilation of the number of mutation types. For instance, in some instances the vector may contain the counts of the 96 mutation types concept from Alexandrov et al, “Signatures of mutational processes in human cancer,” Nature, 2013, the content of which is incorporated herein by reference in its entirety. These 96 mutation types include 5′ flanking base (A, C, G, T), the 6 substitution classes (C>A, C>G, C>T, T>A, T>C, T>G) and 3′ flanking base (A, C, G, T) leads to a 96 mutation types classification (4×6×4=96). Additionally, other mutational signatures could be developed over different types of mutations such as genomic rearrangements.

After determining the mutational spectrum of the sample, it may be compared to predetermined clusters of mutational spectrums 240. The predetermined clusters of mutational spectrums are derived by determining mutational spectrums from the whole genome of various samples, and clustering the samples using, e.g., hierarchical clustering, based on the fractional occurrence of each mutation in a sample. In other examples, the predetermined clusters may be determined from samples that have less than the whole genome sequenced (e.g. a subset of the genes as described above) and using different clustering methods including k-means clustering, silhouette width, expectation maximization, etc.

The sample mutational spectrum may be compared to the predetermined clusters using a variety of methods including a likelihood similarity measure 245 as disclosed herein. Additionally, other methods may be utilized including a likelihood calculated with different probability distributions rather than a binomial distribution (e.g. negative binomial) or a measure other than likelihood such as cosine similarity or Euclidean distance. Then a matching cluster(s) may be identified 250.

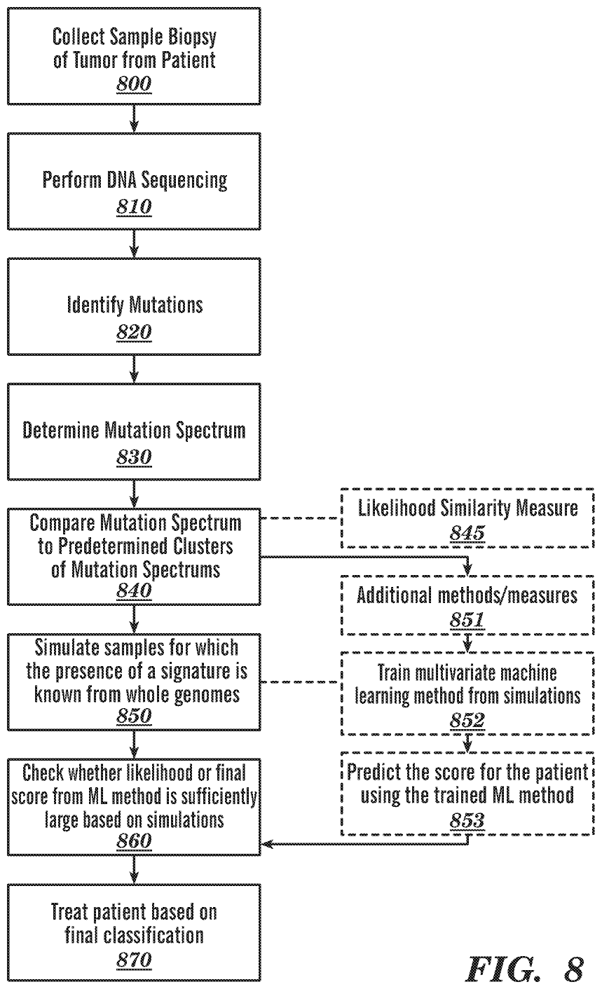

In other examples, and as depicted in FIG. 8, a more detailed procedure can be followed, initial steps 800-845 being identical to steps 200-245. WGS data may be down-sampled to the regions covered by targeted gene panels to simulate panel data 850. The simulation serves multiple purposes, the primary purpose being determining a threshold that defines a sufficiently large matching score that yields few samples that are falsely matched 860.

In other examples, additional matching scores such as cosine similarity can be calculated to a signature in the catalog and the magnitude of a signature can be calculated with linear decomposition (NNLS) to find magnitude of several signatures simultaneously 851. These are standard methods that are effective when the number of mutations is large, but they can improve the robustness of the method when used in combination with matching to a cluster. A multivariate machine learning (ML) model can be trained that combines several features including the matching score to clusters and predicts a final score 852. Simulations may be used in the training.

In other examples, the training can be done using panel data or simulated panels from other sources rather than WGS data, if the status of the signature is known by other identifiers rather than the analysis of WGS data.

The trained ML method may be used to predict a final score that indicates presence of a specific signature for which the training has been done 853.

For instance, a trained gradient boosting machine(s) may be utilized to combine the above features or different combinations of the above features to output a final score 852-853. All measures, including likelihood measures, can be calculated in simulations mentioned above, and can be combined to output a final score using machine learning methods. For instance, a gradient boosting machine could be trained using simulated spectrums and samples from the publicly available whole genome sequenced data 852. In other examples, other types of machine learning algorithms such as random forest, naiive Bayesian, elastic net, support vector machines, lasso, and generalized linear regression could be utilized to analyze the features.

In some examples, the features that could be combined into a single score include:
(1) cosine similarity;
(2) likelihood similarity measures for signature positive and signature negative clusters;
(3) signature exposure calculated with NNLS;
(4) likelihood of a given NNLS decomposition compared to other possible decompositions; and
(5) total nNumber of SNVs.

These features could be combined with a gradient boosting classifier to apply the appropriate weighting to the features. In some examples, certain subsets of the features may be more important. For instance, for panel based data the likelihood similarity measures may be most important or the only features utilized. For WGS data, the linear decomposition features may be the most important but linear decomposition features may not be accurate for panel data (with much smaller numbers of mutations).

The output score may be utilized to determine whether a patient is likely positive for certain defects or maladies associated with particular signatures 260/870. Accordingly, different score thresholds may be set based on the confidence required or desired based on the anticipated action (e.g. treatment). For instance, if a drug with low side impacts is available, the threshold may be set lower and the drug administered as a prophylactic. In some instance, more aggressive treatments could be utilized if there is a higher confidence based on the resulting score. Having a higher confidence may also be more optimal in order to observe a better response to treatment in the selected cohort because of the higher specificity.

Additionally, a caregiver may treat the subject 100 based on the final classification 270/870. For instance, the patient may be treated with a PARP inhibitor or Pol theta inhibitor if the mutational signature relates to homologous recombination deficiency. Also, the patient 100 may be treated with any other suitable treatment targeting homologous recombination deficiency if the mutational signature relates to homologous recombination deficiency.

### EXAMPLES

The following examples are provided to better illustrate the claimed invention and are not intended to be interpreted as limiting the scope of the invention. To the extent that specific materials or steps are mentioned, it is merely for purposes of illustration and is not intended to limit the invention. One skilled in the art may develop equivalent means or reactants without the exercise of inventive capacity and without departing from the scope of the invention.

### EXAMPLE 1

### Sigma Algorithm

Existing methods for signature analysis follow one of two approaches. One approach is to discover signatures from all available genomes by applying an unguided decomposition algorithm, such as non-negative matrix factorization (NMF) (Alexandrov et al., 2013b; Blokzijl et al., 2018; Gehring et al., 2015) or its Bayesian counterpart (Kasar and Brown, 2016; Rosales et al., 2017). The other approach is to find an optimal combination of pre-defined signatures for a given sample, e.g., by minimizing the difference between the compound spectrum and the observed spectrum using non-negative least squares (NNLS) (Blokzijl et al., 2018; Huang et al., 2018; Lee et al., 2018; Rosenthal et al., 2016). The commonality in the two approaches is the decomposition step in which the mutational spectra of tumors are described as a linear combination of signatures. In the first case, the signatures are discovered simultaneously with their coefficients, which we also refer to as ‘exposures’, in the second case, a set of signatures is given, and the algorithm determines their exposures.

These methods, however, are inadequate when the number of mutations is small. The NMF approach is unguided and therefore requires more information than the latter. When there is insufficient information—i.e., not enough genomes or not enough mutations per genome—only a subset of signatures that cause high mutational burden or are active in the vast majority of genomes are discovered, leading to low sensitivity. Moreover, the spectrum of a single signature is often affected by other signatures active in the same dataset, e.g., signatures with correlated exposures may not be separated into distinct components (see Supplementary Methods Section 1 for other computational issues). The second approach, on the other hand, cannot do be used for de novo signature discovery, and it requires the user to select the signatures to be used in the decomposition based on prior knowledge. If it is not constrained, it frequently leads to misidentification of signatures (low specificity) because the optimal solution may not be unique when there are many signatures in the catalog but only few mutations. In Supplementary FIG. 1, the pitfalls of these two approaches and how we address them are illustrated.

SigMA algorithm Example Overview

One proposed example approach is the SigMA algorithm (Signature Multivariate Analysis) that enables accurate identification of mutational signatures even when the mutation count is very small. It combines the elements of the approaches above with novel measures for associating mutations to signatures. First, it replaces the spectrum decomposition step with a more robust clustering step. This is possible by utilizing a rich resource of existing WGS data that informs us on the co-occurring signatures and their relative contributions for a given tumor type and/or subtypes.

In some examples, after identifying clusters of samples with similar mutational spectra for each tumor type (see Online Methods and Supplementary Methods Section 2), the system compares the mutational spectrum of a new sample to each of the ensemble averages using the likelihood-based similarity measure described below. This allows the classification of the new tumor together with tumors that share similar combinations of signatures. When the mutation count is small, this is a more stable approach for inferring a combination of signatures present in the sample than performing a linear composition directly (see Supplementary Methods Section 8).

In one example, SigMA consists of 3 main steps. First, mutational signatures in WGS data are discovered using NMF, and tumor subtypes based on signature composition of tumors are determined. These subtypes are used as a reference for panels. SigMA contains the WGS analysis results, and this step may not be repeated when the tool is used (Supplementary Methods Sections 1-2). Second, for each new sample, the novel likelihood measure (Supplementary Methods Section 4), cosine similarity (Supplementary Methods Section 3) and exposure of Signature 3 with NNLS (Supplementary Methods Section 5) are calculated. Third, trained gradient boosting machines specific for each tumor type, determine a final score using the features from step (2) as an input.

Clustering tumors by signatures to define tumor subtypes: In some examples, microsatellite-stable (MSS) tumors are clustered based on the fractions of signatures and the existence of Signature 3 (a feature that takes values of 0 for tumors without Signature 3, and 1 for tumors with Signature 3), using hierarchical clustering (FIG. 1a, Supplementary FIG. 2a). To choose the number of clusters, the within-cluster sum of squares and between-cluster sum of squares are calculated. Tumors with microsatellite instability (MSI) are clustered separately following the same procedure (Supplementary FIG. 2a). For each tumor type, the average mutational spectrum of MSS tumors is calculated and combined with the tumor type-independent average spectrum of MSI tumors, and also a tumor type-independent average spectrum of hypermutated tumors with POLE exonuclease domain mutations.

Likelihood calculation: Likelihood is the probability for observing a set of mutations for a given underlying mutational signature or mutational signatures, which define the underlying multinomial probability distribution. Multinomial distribution is a generalization of a coin flipping example (discussed in detail in Supplementary Methods Section 4d). Shortly, the number of mutations are equivalent to number of times the die is rolled, and the die has 96 faces instead of 2. To associate the observed mutational spectrum to one of the possible underlying spectra, likelihood of all the possibilities are calculated and are normalized to yield 1. Trying to infer the underlying mutational signature from observed mutations is similar to having more than a single coin but several coins with different H and T probabilities and attempting to tell which coin was flipped based on the observed H and T counts. A formal description can be found in Supplementary Methods.

Simulations for tuning and testing the multivariate model: To tune the multivariate model and to test its performance, it is necessary to have a set of panels, for which we know the truth about the presence of Signature 3. In another study (Davies et al., 2017), in which the HR defect is identified from WGS data, the tumors with bi-allelic inactivation of BRCA1 and BRCA2 were used as a true positive set of HR defect. However, as HR defect is more prevalent than BRCA1/2 mutations. In that example, even if the positive set is defined carefully the negative set can still contain samples with HR deficiency due to other causes than BRCA1/2 mutations. The disclosed systems and methods, in one example, used the WGS NMF results as a reference, and simulated panels from WGS data and truth positive and negative set is defined based on the Signature 3 status in WGS data. The simulations were done by downsampling the WGS data to the target regions of the panels [refs]. However, it was found that the difference in depth of coverage between the WGS (˜40×) and panel (˜1000×) resulted in a smaller number of mutations in the simulated panel, compared to the original panel datasets. Therefore, the number of mutations were increased in the panel simulations from the WGS data by randomly sampling the mutations from the whole exome region. The amount of additional mutations added in this way and how the effects of differences were determined in coverage are discussed in Supplementary Methods Section 11.

The SigMA code and detailed documentation are available at https://github.com/parklab/SigMA.

### Application to Breast Cancer

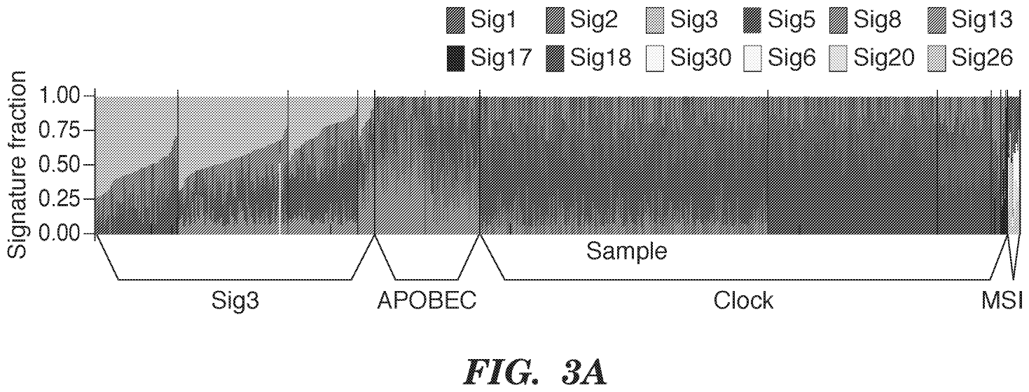

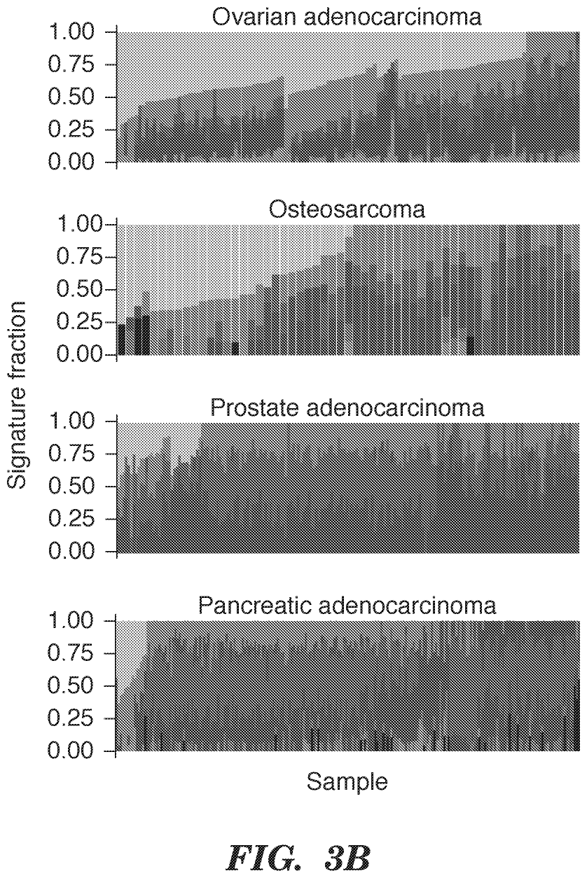

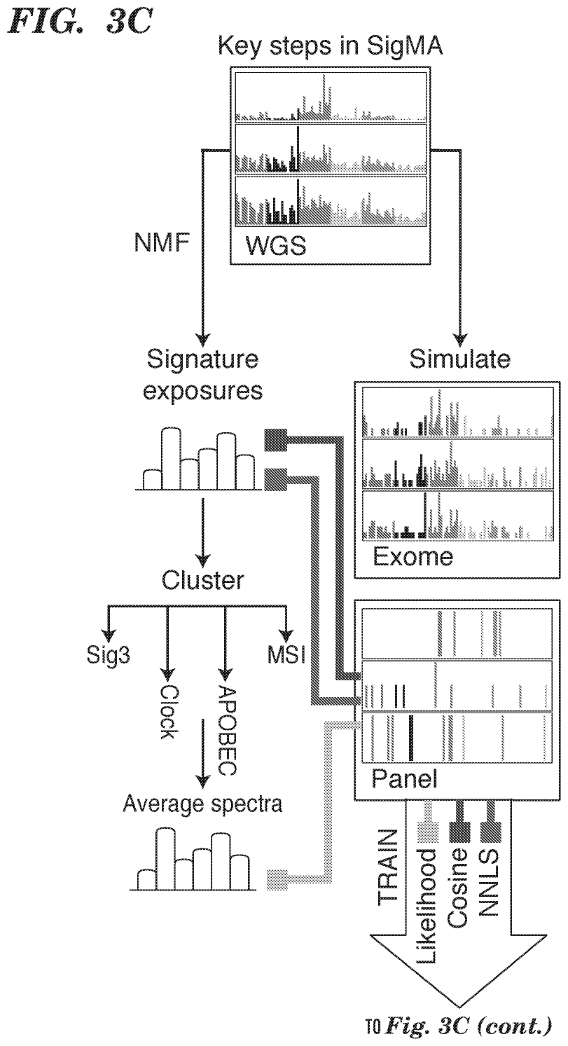

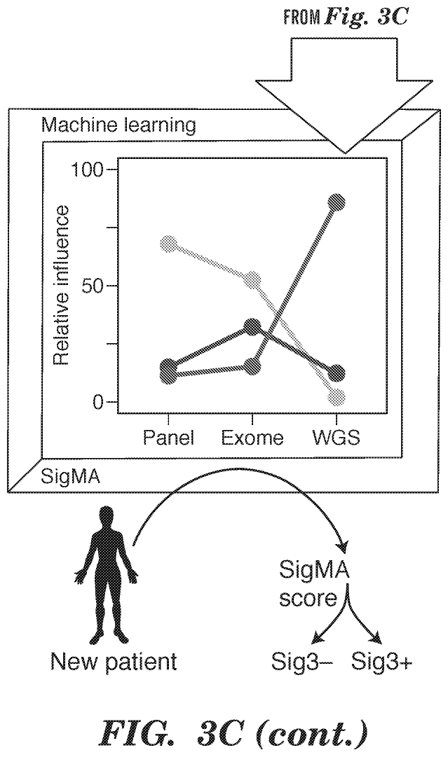

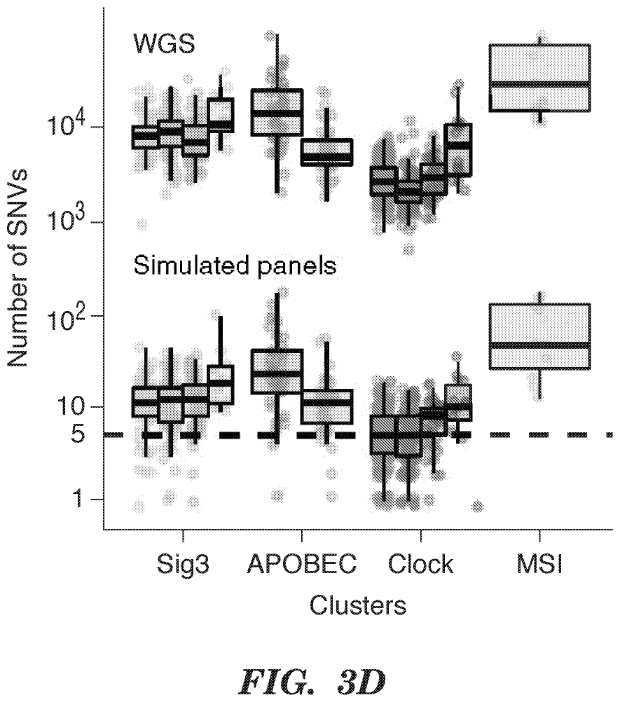

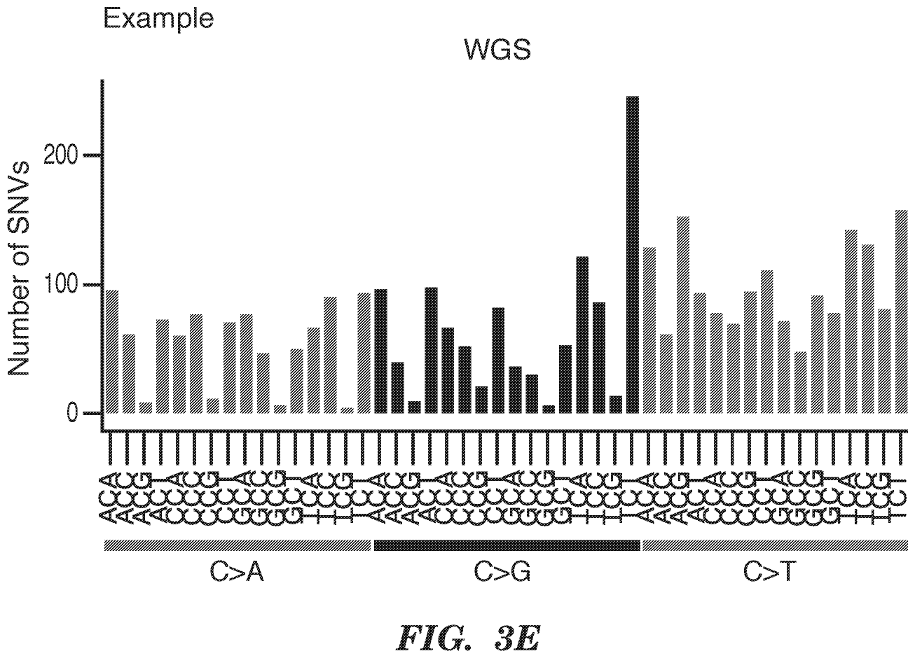

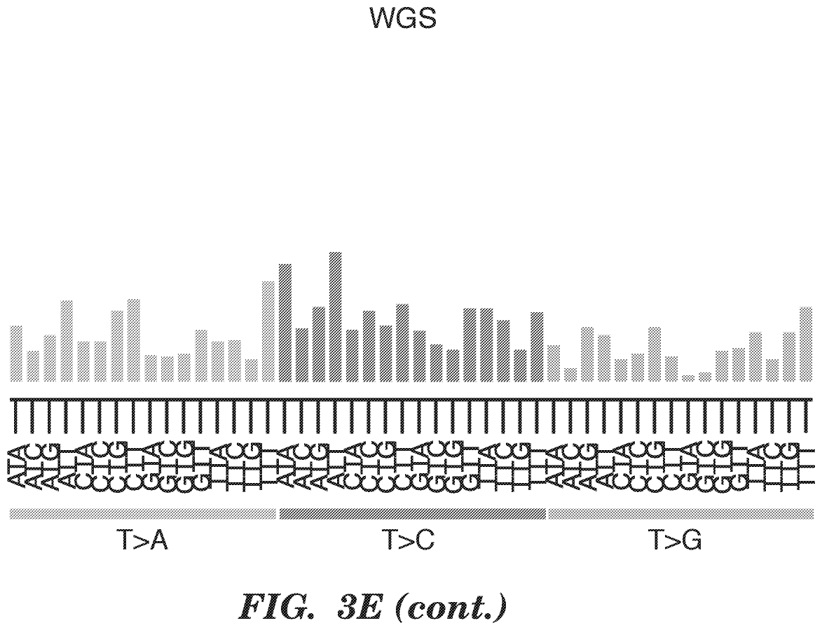

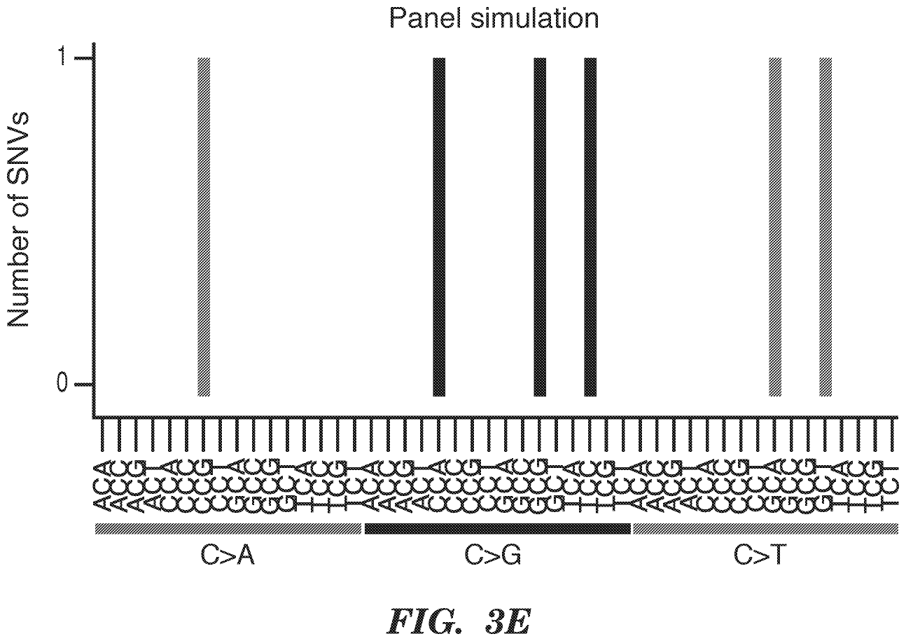

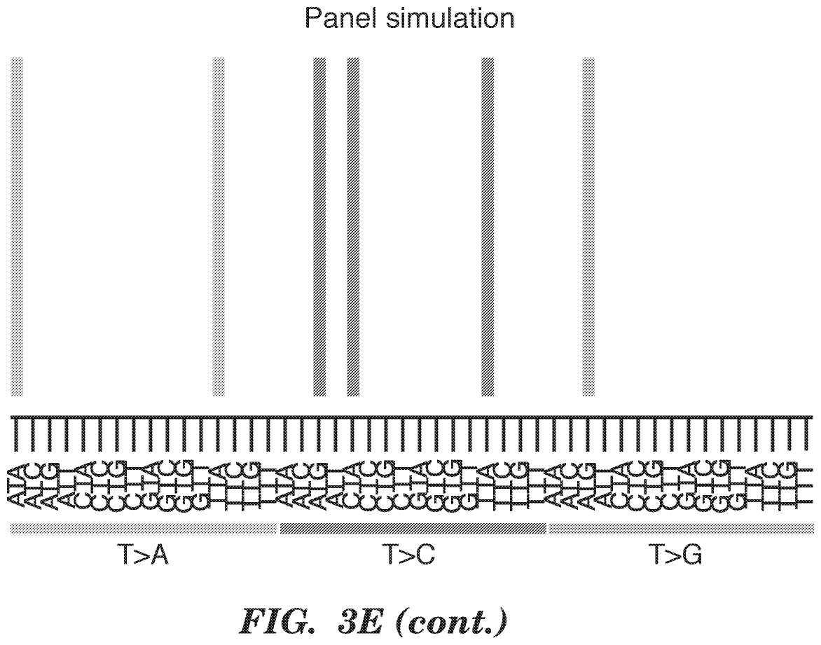

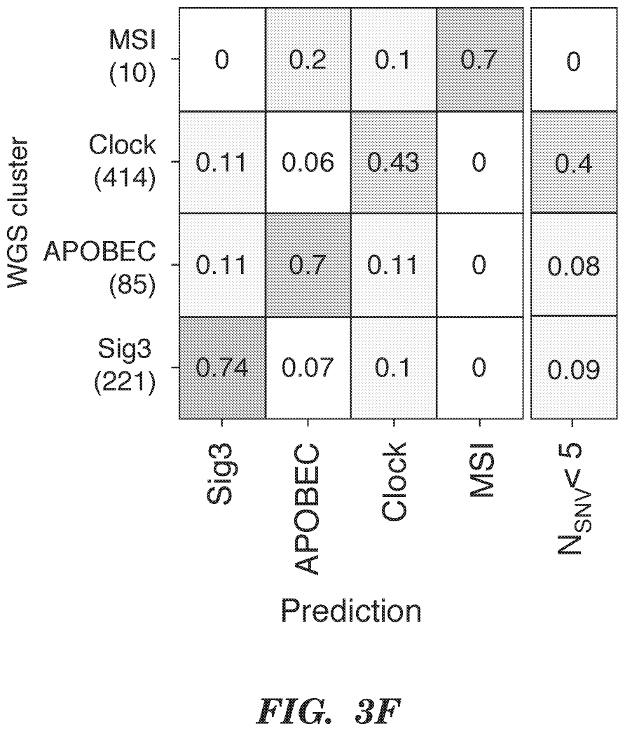

In this example, the disclosed systems and methods were applied to breast cancer, and 731 WGS samples were utilized, of which 67 (9%) had bi-allelic inactivation of BRCA1/2. 12 clusters were obtained as shown in FIG. 3A. These clusters fell broadly into five categories: Signature 3-positive, predominantly APOBEC (Burns et al., 2013; Kazanov et al., 2015; Nik-Zainal et al., 2012; Roberts et al., 2012), dominant ‘clock’ (Alexandrov et al., 2015), microsatellite instability (MSI; this included some non-breast cases), and the rest. Based on these clusters, the system classified a new sample to be, e.g., a Signature 3-positive when the most similar ensemble average was the Signature 3-positive group. The results of clustering in some other tumor types are shown in FIG. 3B and Supplementary FIG. 2a; the differences among them support the need for a tumor type-specific procedure.

Another example component of SigMA is the cosine similarity measure used for matching the mutational pattern of a given sample to the ensemble profiles. A standard measure for comparing two spectra has been the cosine similarity, which is the cosine of the angle between two vectors in space. This measure is flawed in that it is sensitive to minor changes in the mutational spectrum when the mutation count is small; even a single mutation can cause a large deviation in the angle. Accordingly, the disclosed systems and methods utilize a much more robust and statistically sound approach: calculating the likelihood of the mutations in the new sample to be generated from the probability distribution defined by the mutational profiles of each tumor cluster (see Online Methods and Supplementary Methods Section 4). A simple coin-tossing example that illustrates the differences between the two methods is in Supplementary FIG. 3.

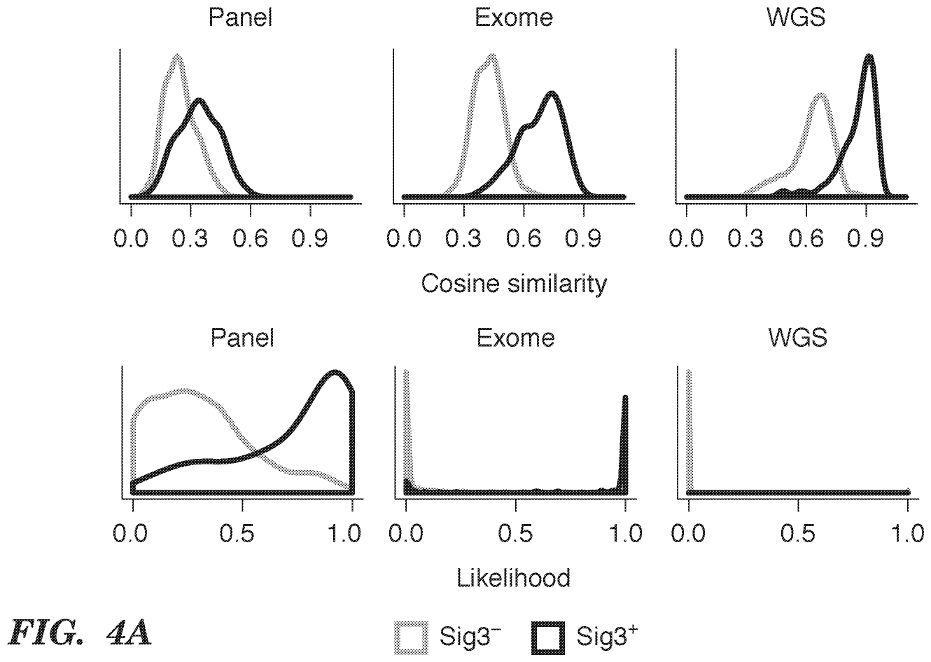

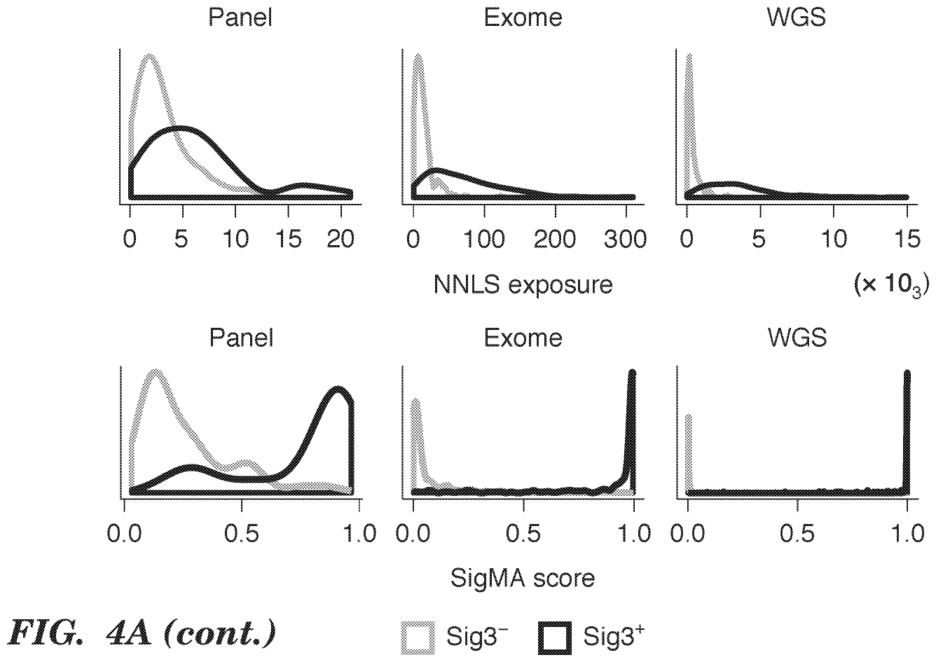

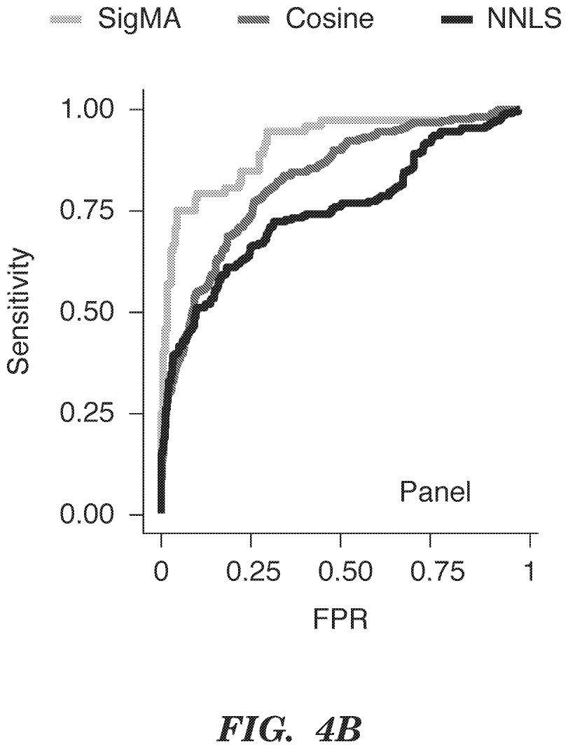

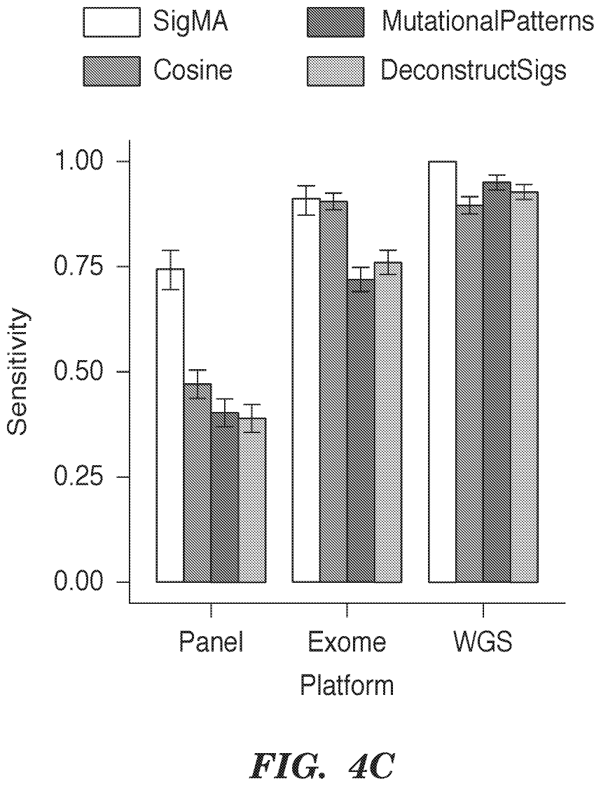

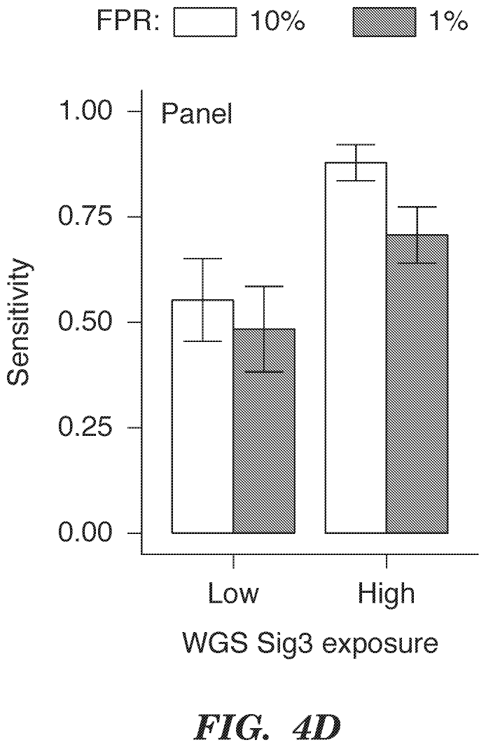

To develop a unified platform that can be applied equally well to different types of sequencing data, in one example, the disclosed systems and methods combine several variables commonly used in signature analysis with our novel likelihood measure in a multivariate form as illustrated in FIG. 3C. Thus, whether the most informative measure is the likelihood calculated from average spectra (for panels) or linear decomposition accompanied with likelihood (for WGS), our platform handles it automatically, with the weighting of different components handled using Gradient Boosting Machines (Supplementary FIG. 4c; see Supplementary Methods Section 7).

### Application of SigMA to Simulated Panel Data

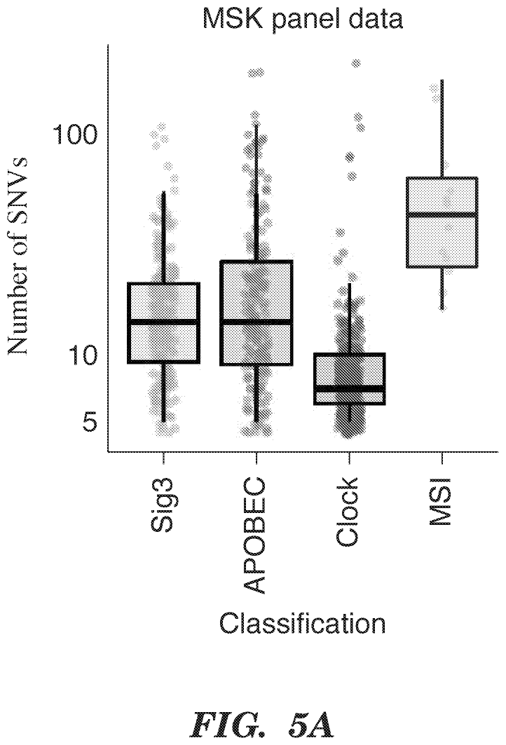

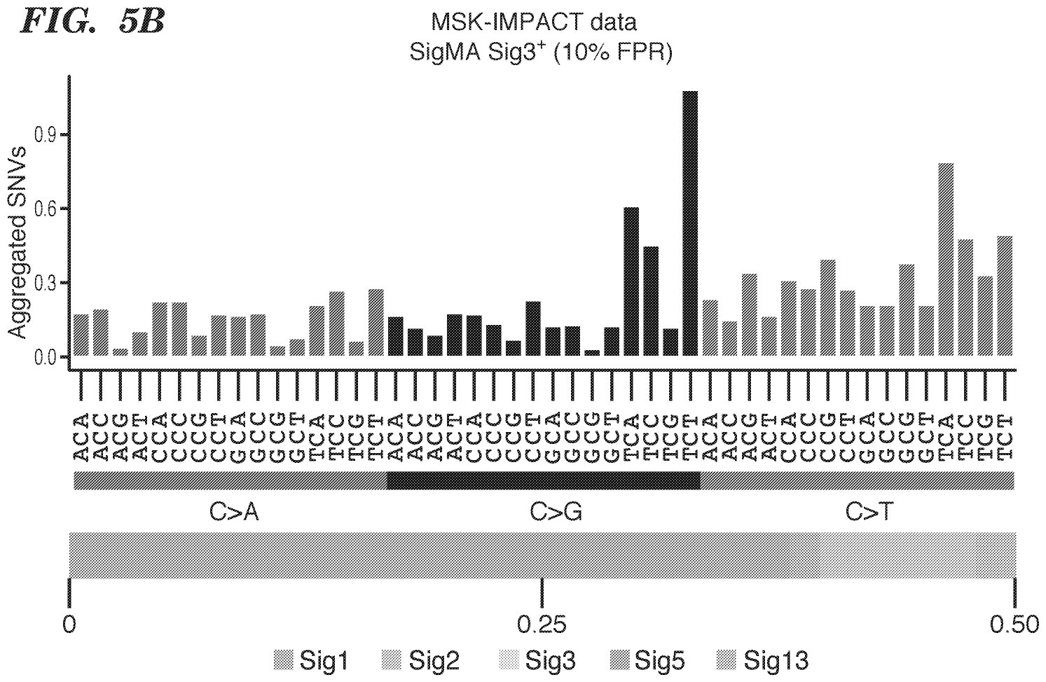

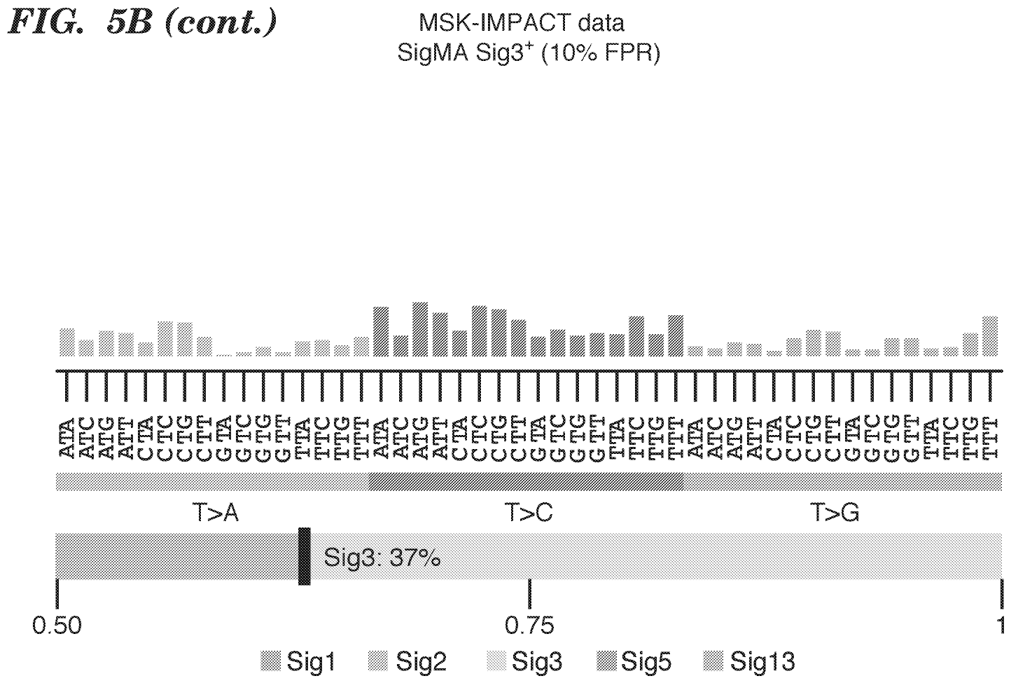

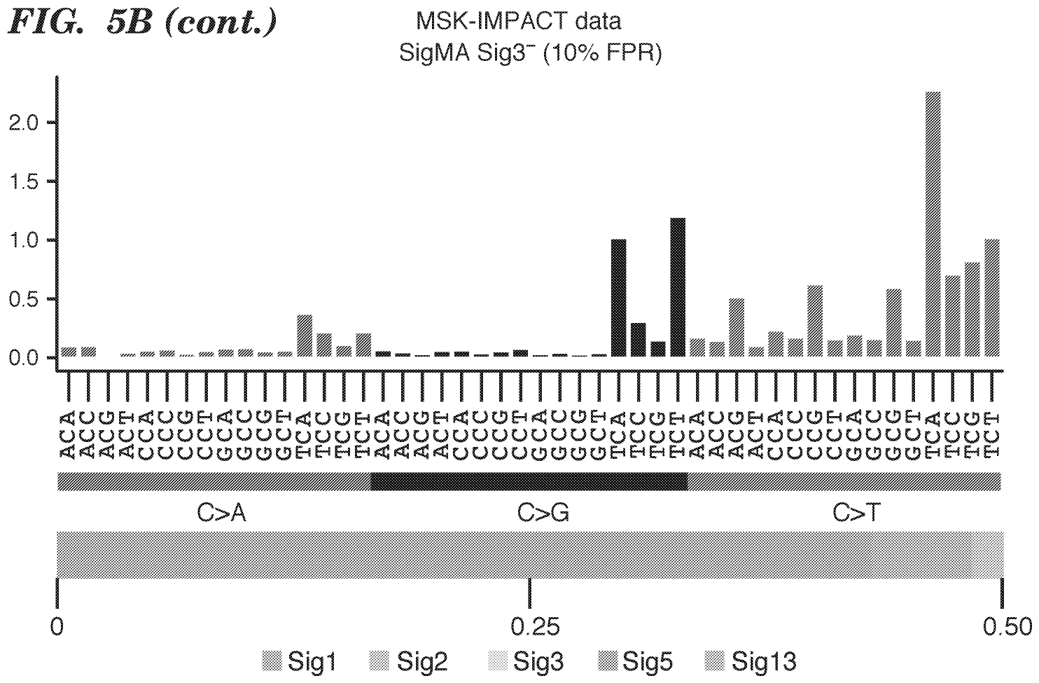

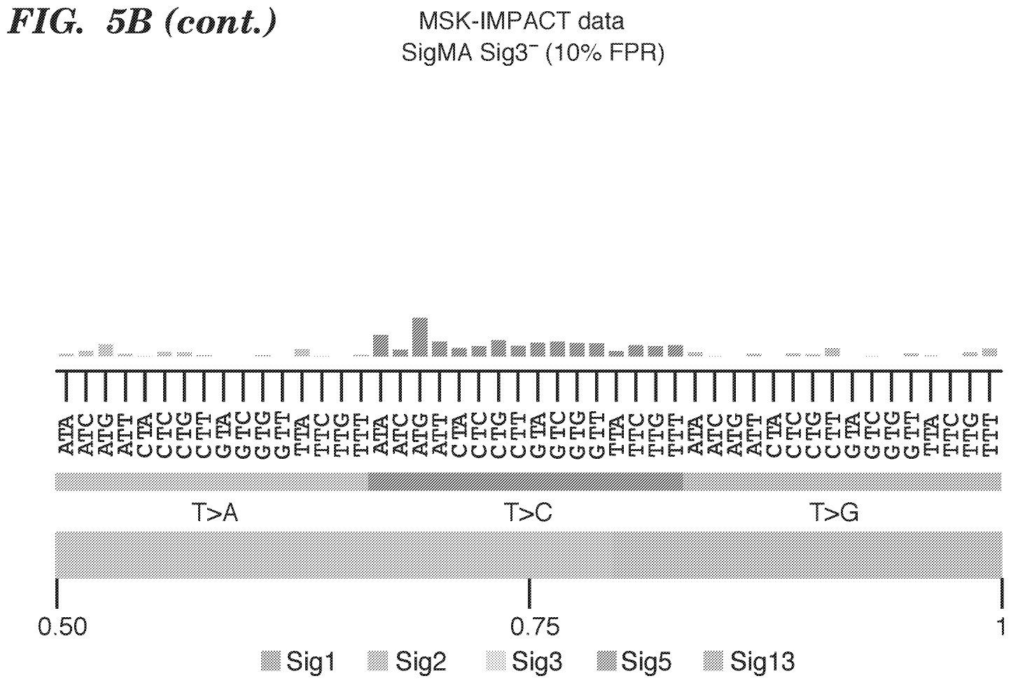

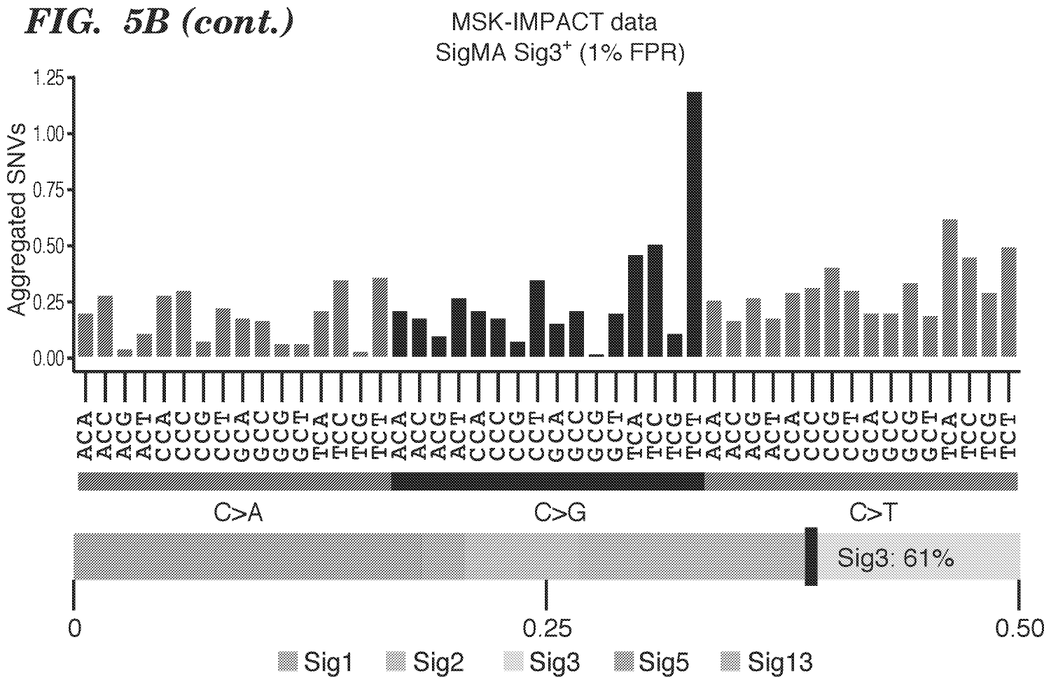

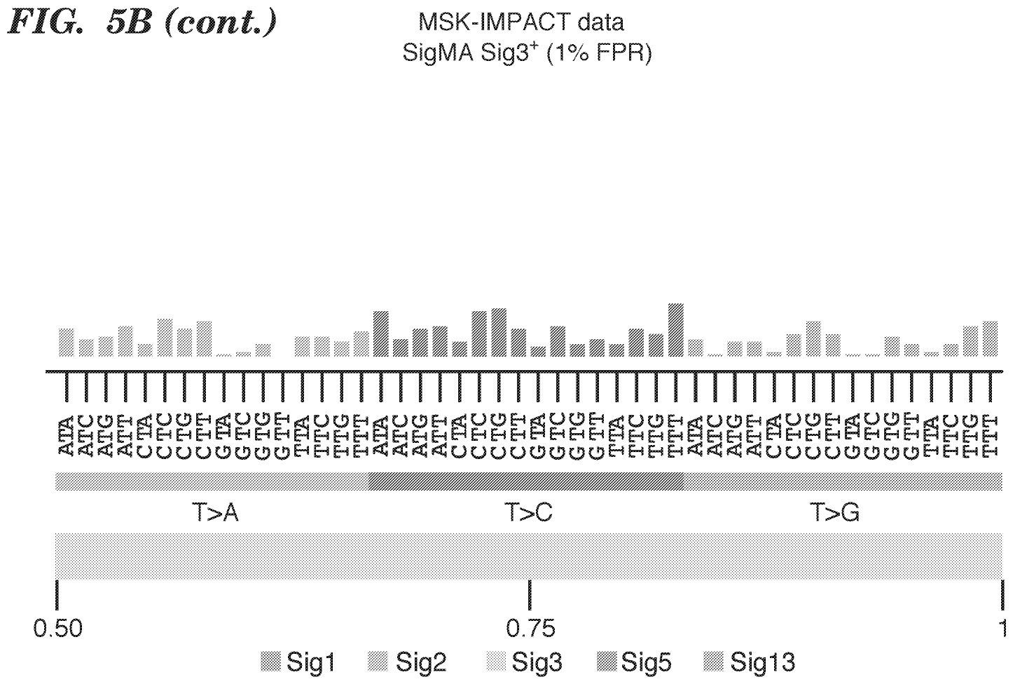

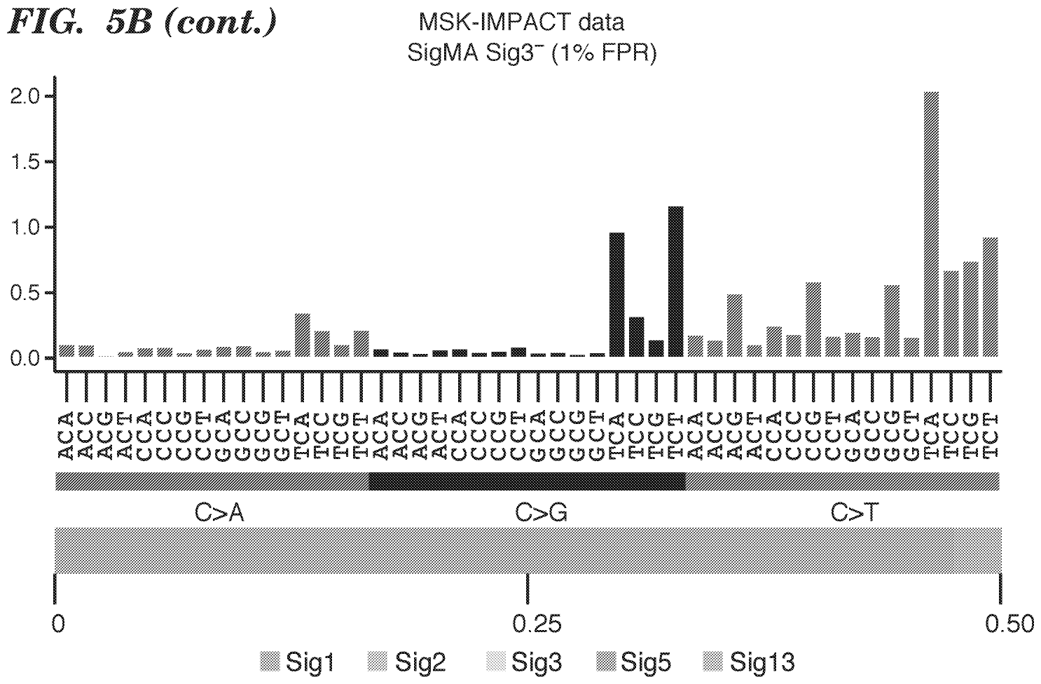

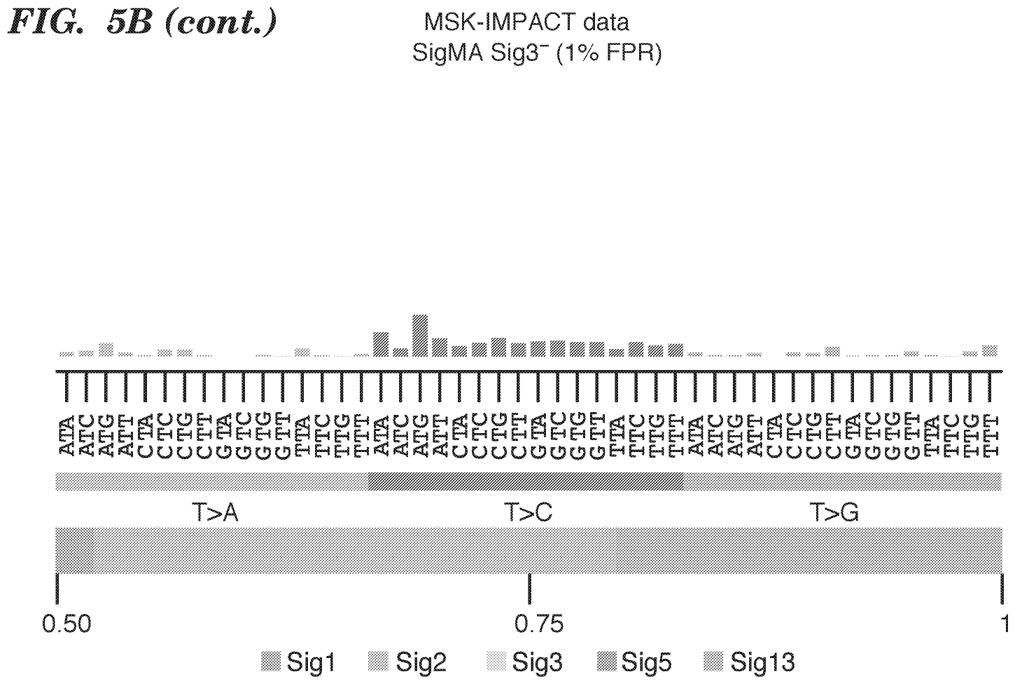

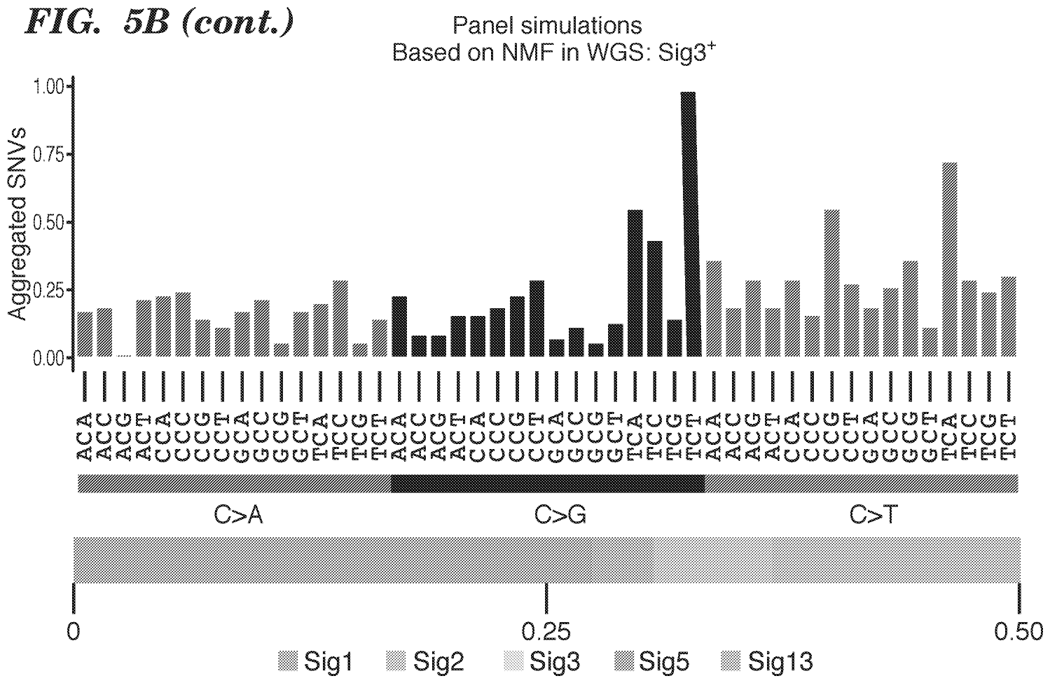

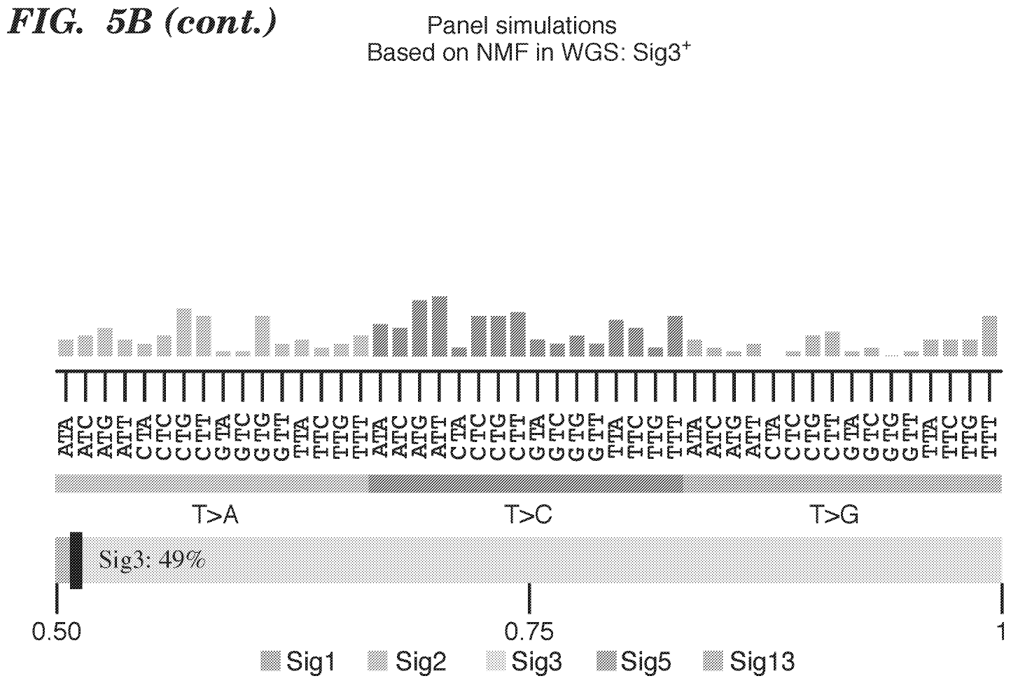

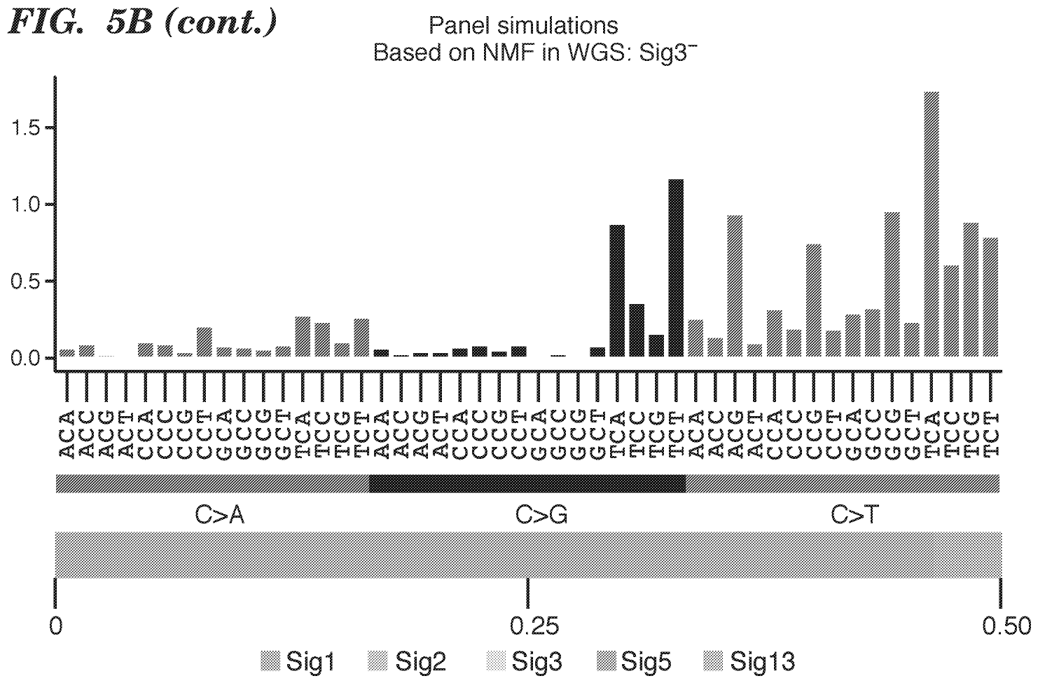

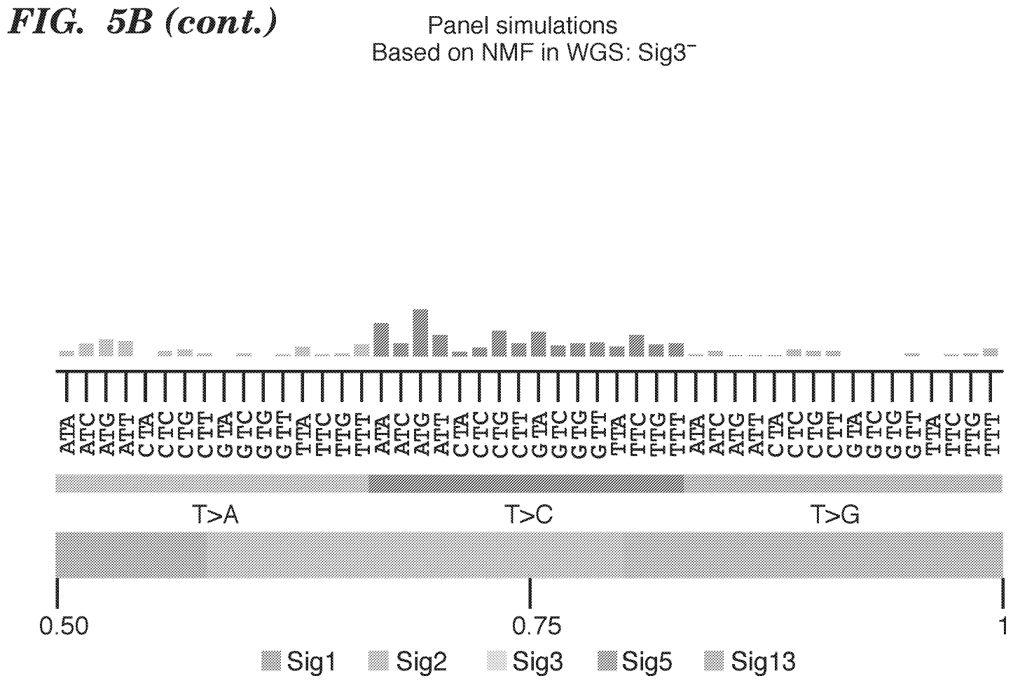

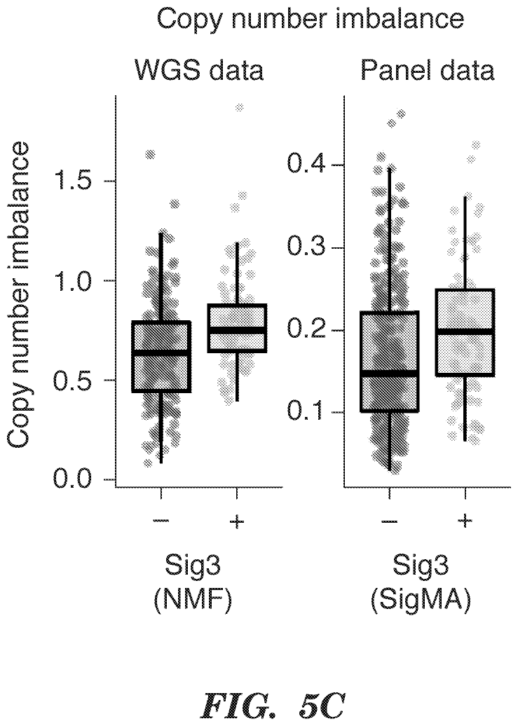

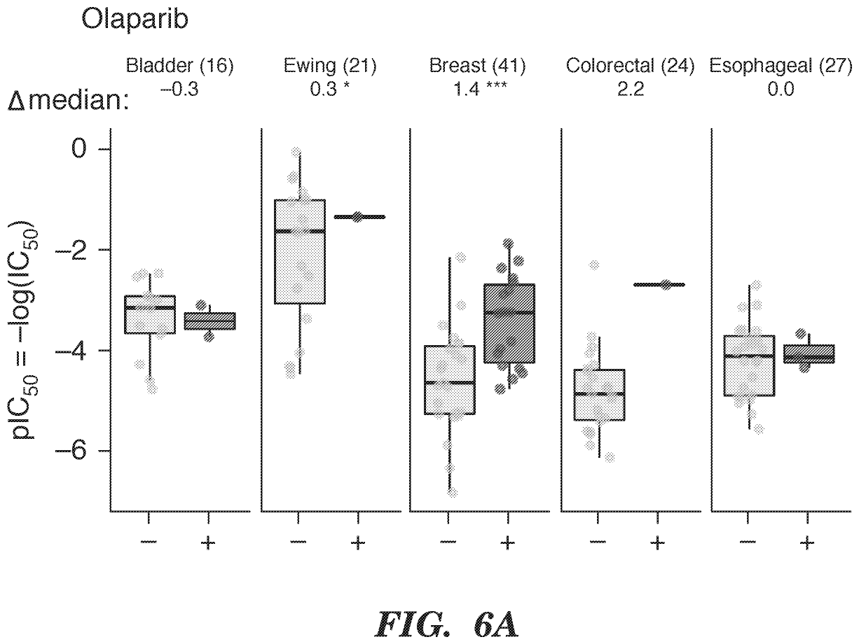

To illustrate some of the advantages of SigMA, simulated datasets were generated that mimick two widely used panels, MSK-IMPACT (Zehir et al., 2017) and FoundationOne (Frampton et al., 2013). The simulation was performed by down-sampling from the 731 WGS samples, whose signature decomposition will serve as the gold standard (detailed simulation processes including adjustment for read depths are discussed in Supplementary Methods Section 11). For a 410-gene panel covering 2.36 Mb capture region (MSK-IMPACT), the number of mutations is typically reduced by ˜1000-fold (FIG. 3D); the distribution of mutation counts is similar to that observed for real data (see next section; Supplementary FIG. 5a, Supplementary FIG. 9c). Among the 221 Signature 3-positive samples, the average mutation count is 11.3; 19 (8.6%) had fewer than 5 mutations.

The sparsity of the simulated mutational spectrum for a Signature 3-positive tumor (FIG. 3E in contrast to the WGS case in FIG. 3F) illustrates the difficulty of making inferences about mutational signatures using panel-derived mutation counts: panel data have much smaller mutation counts spread over the 96 triplets, with many having 0 or 1 mutation. Under these conditions, SigMA correctly classified these samples as Signature 3-positives, whereas cosine similarity or NNLS were not sufficient informative and predicts another signature (FIG. 3E). The SigMA score for Signature 3 is driven by likelihood (˜70%) and the simulation indicates that this score corresponds 1% false positive rate (see Supplementary Methods Section 7). The signature composition of the matched ensemble from WGS reference is strikingly similar to that of the WGS data from which the panel was sampled (Supplementary FIG. 5g).

When applied to all cases (with at least 5 mutations), the SigMA classification of simulated panels for Signature 3-positive and MSI cases mostly agrees with the true categories defined from WGS, despite the large reduction in the number of mutations (FIG. 3G). Classification of APOBEC and clock groups is less concordant, but this is not unexpected, as discussed in Supplementary Method Section 9.

FIG. 4 illustrates the comparison of the performance for SigMA and two popular methods (cosine similarity and NNLS (Lawson and Hanson, 1995)) in detecting Signature 3-positive tumors. Cosine similarity and NNLS show reasonably good separation between the Signature 3-positive and negative cases for WGS data but not for panel data. In contrast, the disclosed likelihood-based method shows much better separation, especially for panel data (FIG. 4a). The multivariate formulation of SigMA results in further improvement for all platforms (Supplementary FIG. 5d,f). The ROC curves for panels illustrated in FIG. 4B show that SigMA achieves higher sensitivity at the same false positive rate compared to other methods. At the false positive rate of 10%, SigMA gives a sensitivity of 74% for panels, which corresponds to a striking 70% increase relative to other methods as illustrated in FIG. 4C.

In another example, the disclosed systems and methods were applied to simulated data based on the FoundationOne panel (315 genes, 253 genes in common with MSK-IMPACT). Due to the lower genomic coverage (1.96 Mb vs 2.36 Mb), the sensitivity was slightly lower (68%). More sensitivity analysis results are shown in Supplementary FIG. 5b-f. Importantly, it was discovered that number of predicted Signature 3-positive cases that do not have bi-allelic inactivation of BRCA1/2 is 2.1-fold larger than those that do, indicating that a substantial number of cases that may benefit from treatments targeting HR deficiency are missed with the current BRCA-based criterion.

It may be desirable to make more conservative predictions in some clinical settings. When the SigMA threshold was increased so that the false positive rate is reduced to 1%, the sensitivity decreased to 50%. However, the cases passing this more stringent threshold tend to have a larger number of mutations belonging to Signature 3 and might be clinically more responsive as the burden of mutational signatures correlates with the success of PARP inhibitor treatment (Sachs et al., 2018). When Signature 3 contributes a large component of the mutations, sensitivity for detection also tends to be substantially higher (FIG. 4D).

### Detection of Signature 3 in MSK-IMPACT Panels

To validate the performance of SigMA on real panel data, it was applied to the 878 breast tumors profiled on the 410-gene MSK-IMPACT panel (Zehir et al., 2017). For tumors with at least 5 mutations, they were classified into the same 5 categories (FIG. 3a). 213 cases were detected (24%) that are likely to be Signature 3-positive, with 112 (13%) passing a more stringent selection criterion.

When all the mutations found in Signature 3-positive cases predicted by SigMA (FIG. 3b, top 2 rows) were aggregated and its spectrum compared to that obtained from panel simulations (FIG. 3b, bottom row), both their mutational spectra and signature composition (bars below the spectra) are very similar. Moreover, Signature 3 is dominant in Signature 3-positive cases and completely absent in the negative cases; those found with 1% FPR threshold have even greater presence (61% vs 37%) of Signature 3. Although this did not have the ‘gold-standard’ set of Signature 3 MSK-IMPACT cases, the label for simulated panel data were derived from the WGS data, and the similarity we observe here indicates that our predictive model and the estimated sensitivity and specificity are applicable to the clinical panels.

Furthermore, the results were examined to determine whether the Signature 3-positive tumors exhibit copy number (CN) imbalance, which is a typical feature of tumors with HR defect. Although CN profiles inferred from panel data this example were much lower in resolution (see Supplementary Methods Section 10), the calculations show that Signature 3-positive tumors have more imbalanced genomes than others (FIG. 5C, p-value=10-5). This aneuploidy observed in the predicted Signature 3-positive tumors supports the validity of the disclosed approach.

### Identification of Signature 3-Positive Cases in Other Tumor Ttypes

Although HR defect has been most closely associated with breast cancers, it can also manifest itself in other tissues, often through mechanisms that have not been clarified yet. For example, one possible mechanism for HR defect recently described is the EWS—FLI1 fusion in Ewing sarcomas (Gorthi et al., 2018). This fusion leads to accumulation of R-loops that prevents the distribution of BRCA1 to double strand breaks, resulting in deficient HR. Thus, in addition to those tumor types known to be associated with HR defect (ovary, uterus, pancreas, etc), many other tissues may exhibit Signature 3.

There are challenges in applying SigMA to panel data from other tumor types. First, some tumor types have very low mutational burden, and some panels may not capture a sufficient number of mutations for inference. None of the simulated panels for Ewing sarcomas and medulloblastomas, for instance, had 5 or more mutations. For such tumor types, a larger panel may be required for detection of Signature 3. Second, in some tumor types, other signatures that accompany Signature 3 may generate most of the mutations. For example, in prostate tumors, clock signatures are very active, making the detection of Signature 3 more difficult even when it is present. Sensitivity ranges from 53% in osteosarcoma to 74% in ovarian cancer at a FPR of 10% (Supplementary Table 1). This will likely improve as more WSG becomes publicly available.

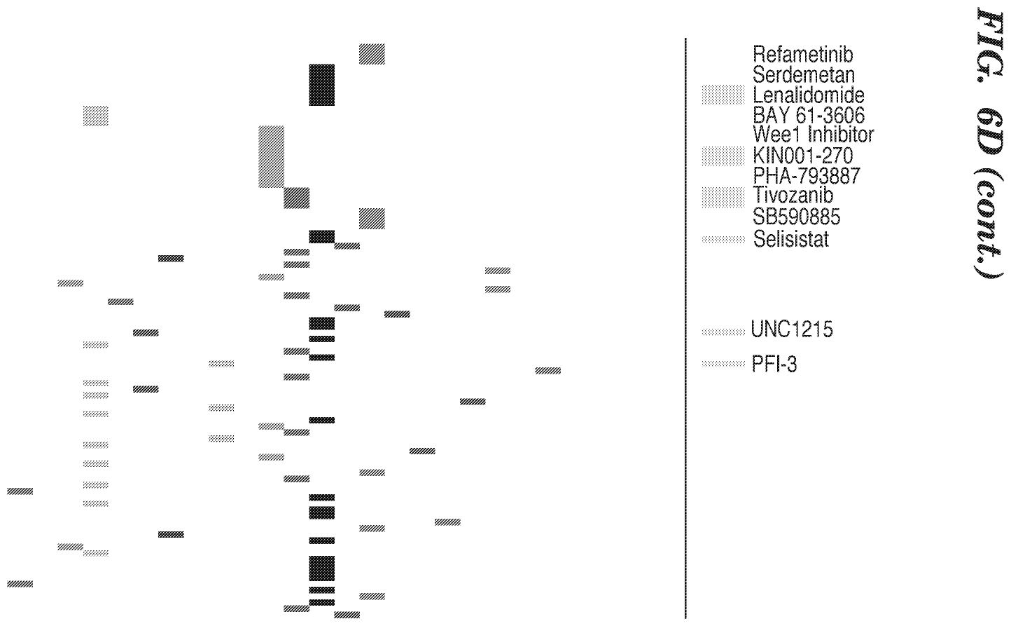

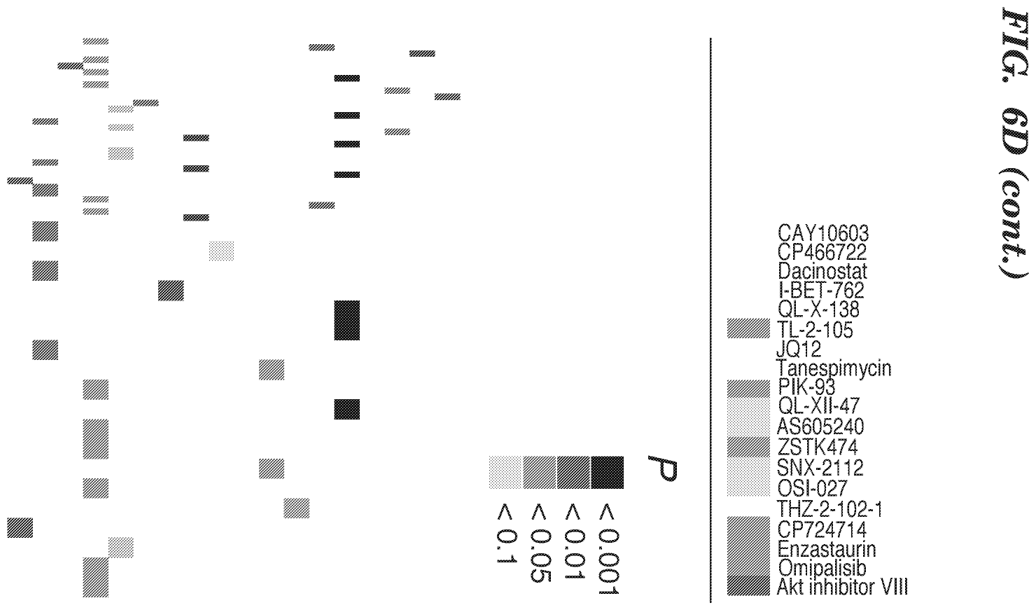

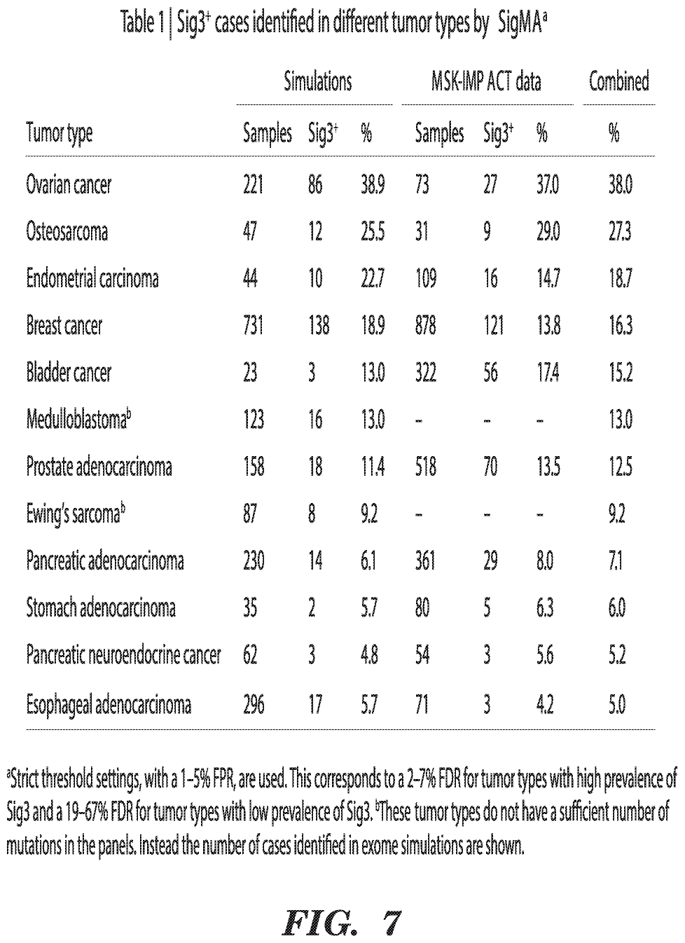

Accordingly, the disclosed SigMA example algorithm detected Signature 3 from panels for multiple tumor types with a frequency ranging from 46.8% in ovarian cancer to 2.3% in esophageal carcinoma (FIG. 7). These values were obtained using the stringent settings of SigMA, with 1% FPR in breast cancer and ranging between 1-5% in other tumor types, to provide a conservative lower bound on the use cases it will have. For the tumor types that have been associated to HR defect in the previous literature—ovarian cancer, uterus corpus endometrial cancer, prostate adenocarcinoma, and pancreatic cancer (Abkevich et al., 2012; Fraser et al., 2017; Ledermann et al., 2012; Waddell et al., 2015; Wu et al., 2018), SigMA identified 46.8%, 14.3%, 11.7% and 7.2% of cases to be Signature 3-positive, respectively. For the other tumor types, such as Ewing sarcoma, osteosarcoma, medulloblastoma, esophagus and stomach adenocarcinoma, the results suggest that 2.3-17.1% of cases are positive for Signature 3.

Response to PARP iInhibitors in Signature 3+ Cell Lines

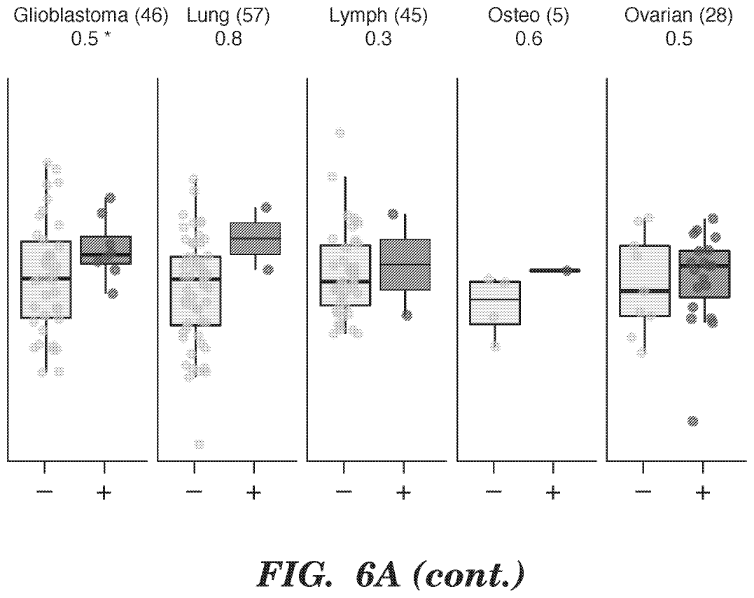

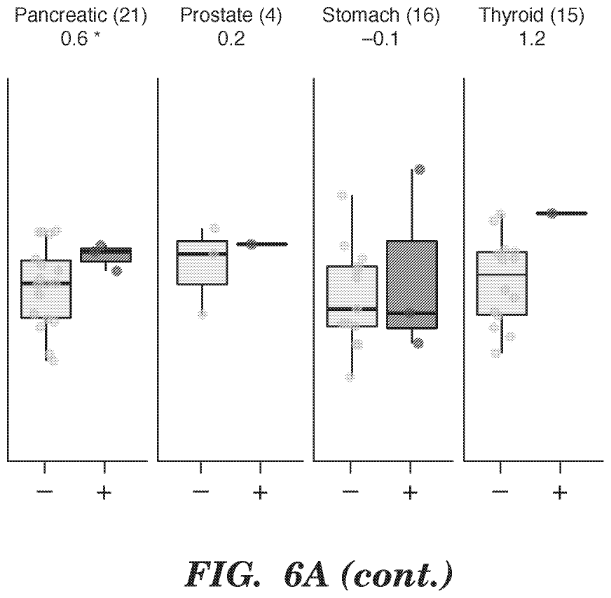

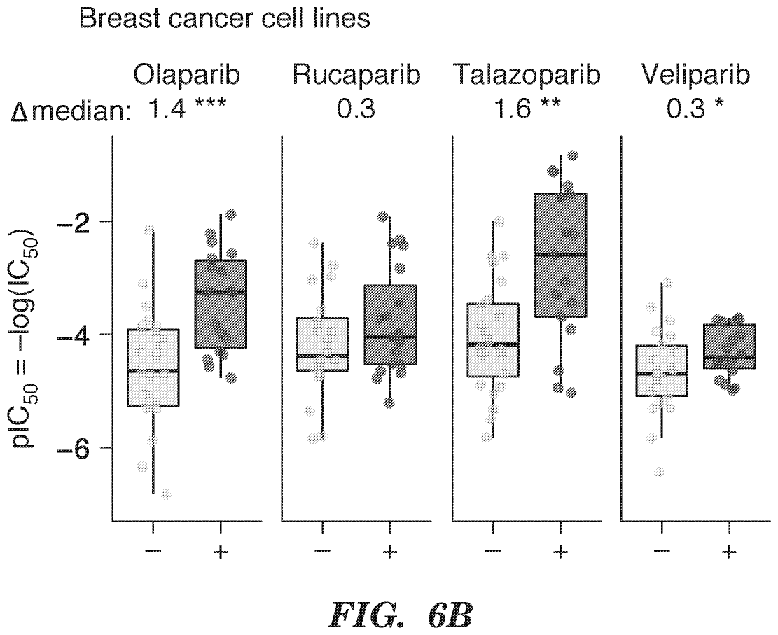

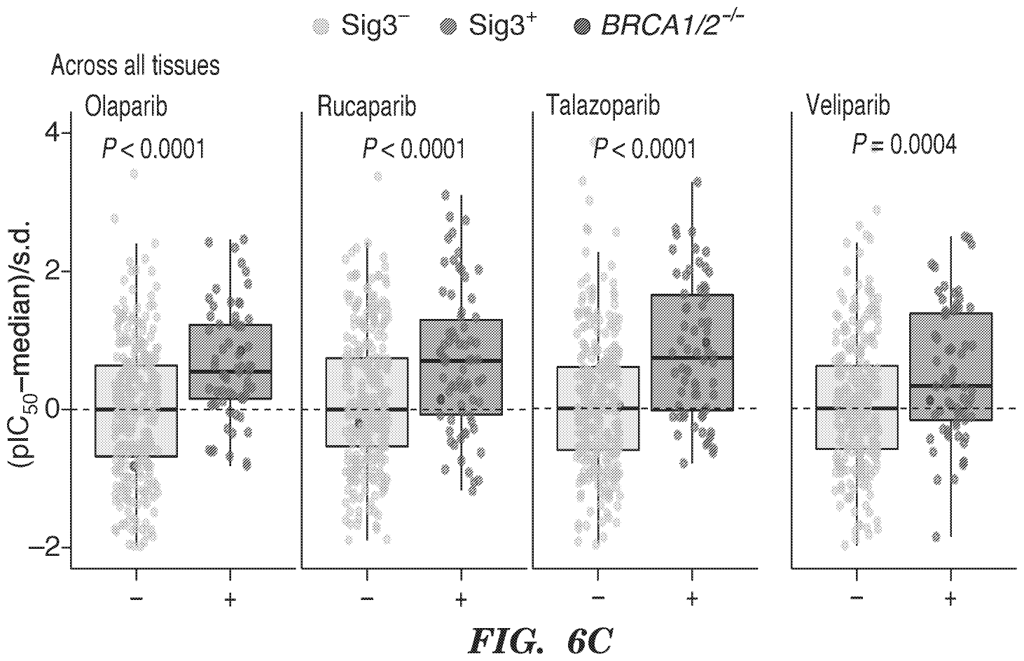

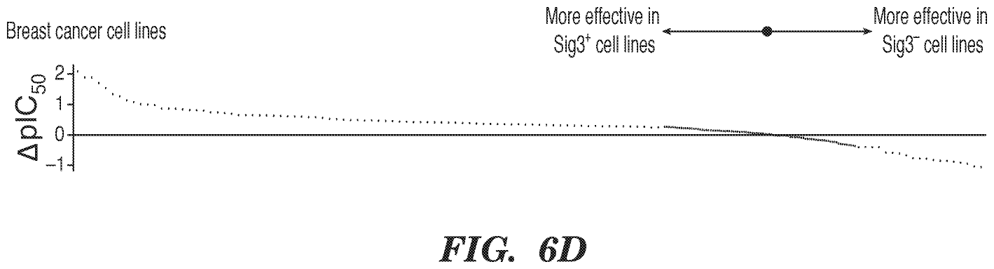

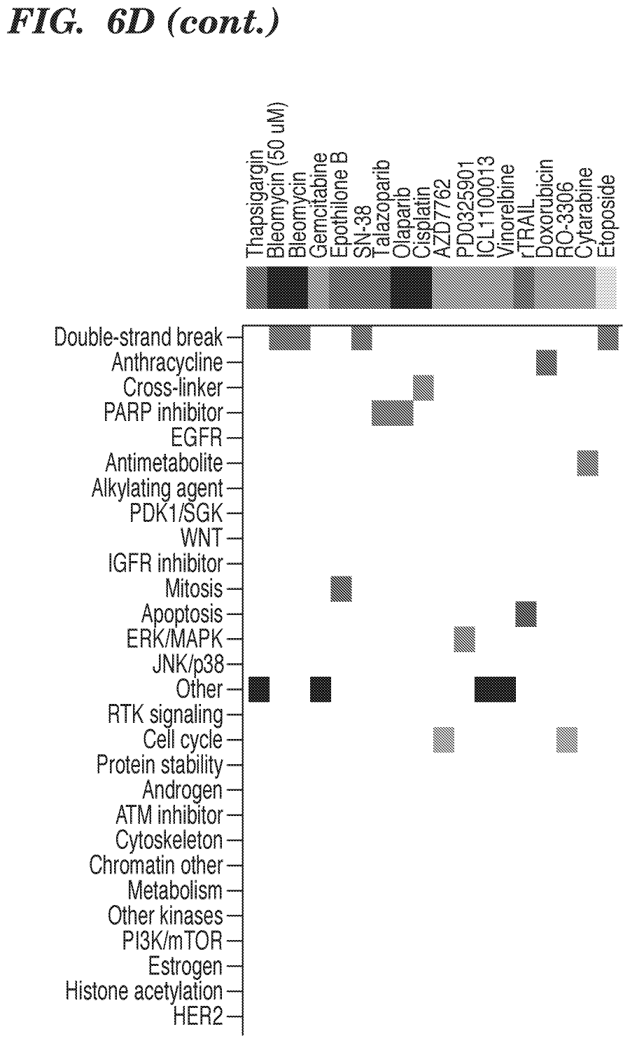

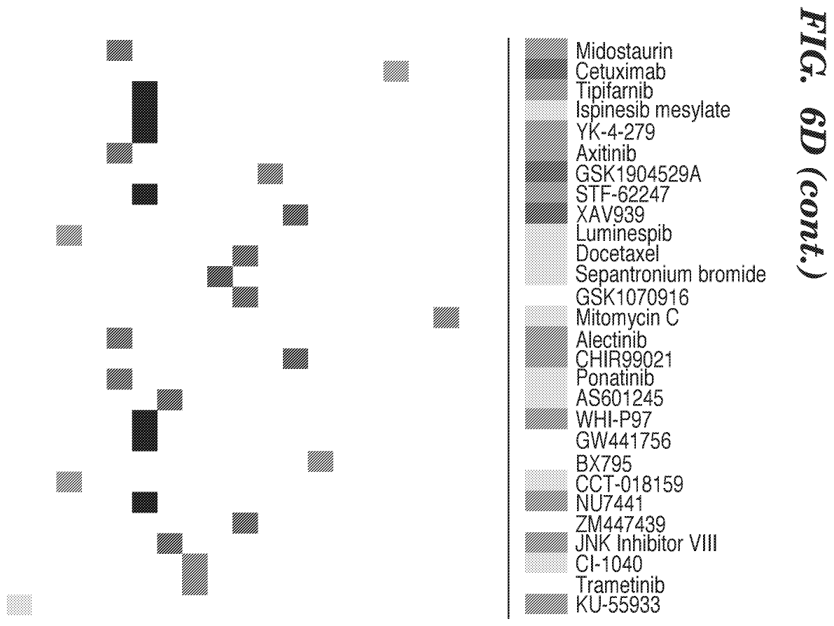

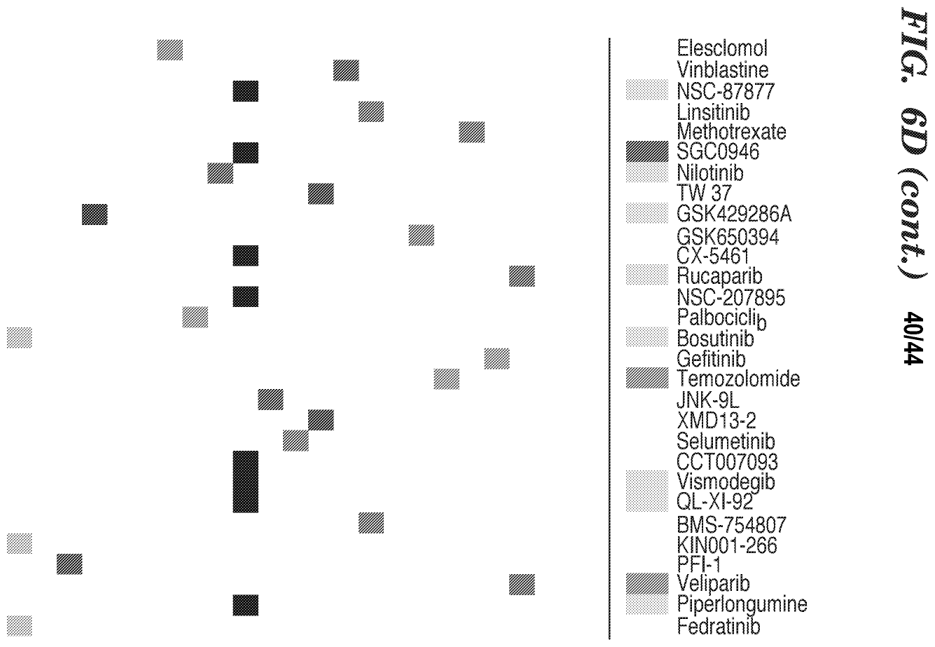

To test the hypothesis that the presence of Signature 3 indicates susceptibility to PARP inhibition, the response of diverse cancer cell lines was examed to the two popular PARP inhibitors olaparib and velirapib (Supplementary Methods Section 12). The disclosed SigMA example algorithm was first applied to 700 cell lines from the Cancer Cell Line Encyclopedia (CCLE) project (Basu et al., 2013) to identify those with Signature 3. Mutation calls from a 1651-gene panel and copy number calls from single-nucleotide polymorphism (SNP) arrays were available for each cell line. In applying SigMA, a stringent filter was used to discriminate the cell lines with Signature 3 mutations from those without Signature 3 but with a large number of in vitro culture-associated mutations (Online Methods and Supplementary Methods Section 12). Also, cell lines with MSI signatures were removed to minimize their confounding effect (see Vilar Sanchez et al. (2009), and Supplementary FIG. 6f). Drug response data was obtrained for olaparib and veliparib on the same CCLE cell lines from the Genomics of Drug Sensitivity in Cancer (GDSC) database, which contains response to 138 anticancer drugs across 700 cancer cell lines (Yang et al., 2013).

FIG. 6 depicts the half maximal inhibitory concentration (IC50) for 85 cell lines corresponding to nine tumor types (more in Supplementary FIG. 6c). For the seventeen breast cancer cell lines, the disclosed SigMA algorithm predicted five to be Signature 3-positive. The IC50 values for olaparib (FIG. 6A) are significantly lower for the five Signature 3-positive cell lines than for the twelve Signature 3-negative cell lines (2.6-fold decrease; p=0.044, Kolmogorov—Smirnov test). For veliparib, the Signature 3-positive breast cell lines again have lower IC50 values, although the fold change is smaller (FIG. 6B). For many tumor types, the number of cell lines is too few for adequate power in tumor type-specific comparison. However, the median IC50 values for are lower for Signature 3-positive samples compared to Signature 3-negative samples in nearly all cases.

When data from all tumor types types are combined (with appropriate normalization to account for different ranges of IC50 values, see Supplementary Methods Section 13), the normalized IC50 values for olaparib are significantly lower for the Signature 3-positive group compared to the controls (FIG. 4c, p-value=10-27). This holds for veliparib as well (FIG. 4d, p-value=10-26). Removing the cell lines with BRCA1/2 mutations (two cell lines with bi-allelic inactivation, two cell lines with a SNV or copy loss on a single allele) from this analysis did not change our conclusion. To ensure that the observed effect is specific to PARP inhibitors, other drugs were examined as controls. The distribution of IC50 values for the Signature 3-positive/negative groups are comparable or have the opposite trend for a set of drugs targeting molecules that are unlikely to synergize with HR defect (Supplementary FIG. 6e).

These results provide experimental evidence for the validity of the disclosed systems and methods in identifying Signature 3 cases and their sensitivity to PARP inhibitors, not only in breast and ovarian tumors but also in other tumor types, irrespective of the mutational status for BRCA1/2.

Drug response in cell-line models: Mutation calls from a 1651-gene capture panel and copy number calls from SNP arrays were available for each cell line from CCLE project and the exome sequences of the same cell lines are available independently by GDSC project. However, in this analysis the whole exomes from GDSC project were not used due to the differences in the spectra of mutational spectra between the CCLE and the GDSC data (Supplementary FIG. 6a-b). The spectra of simulated MSK-IMPACT panels from WGS data of tumors were more similar to the CCLE results. Trinucleotide frequencies alone due to the different target regions of the sequencing platforms for the two projects do not explain the higher C>A and T>G frequencies in the mutational spectra of the GDSC dataset compared to those of the CCLE.

Among 1074 cell lines in total, the mutational spectra of 700 cell lines were analyzed of major tumor types with SigMA, but only 136 of these had drug response data for either olaparib or veliparib. In one example, 74 out of 136 cell lines were used because for the remaining cell lines a different selection for choosing Signature-3 positive tumors was used, as disclosed in Supplementary Methods Section 12.

Detection of SCNA: the consensus structural variation (SV), copy number variation, purity and ploidy datasets were used, which were generated by the PCAWG consortium. The calling pipelines are described in detail in [(i) Dentro, Leshchiner, Haase, Wintersinger, et al. Pervasive intra-tumour heterogeneity and subclonal selection across cancer types; (ii) PCAWG-6. Signatures of selection for somatic rearrangements across 2,693 cancer genomes]. Somatic copy number variations for the MSK-IMPACT data were detected using CNV-kit on hybrid capture panels (Zehir et al., 2017). SNP-array derived copy number information for the CCLE cancer cell lines were downloaded from https://portals.broadinstitute.org/ccle_legacy/home.

### Future Applications in Clinical Practice

Although whole-exome and genome sequencing are commonplace now for exploratory analysis, panel-based sequencing for profiling actionable mutations is predominant in routine clinical settings. Disclosed is the first tool designed to carry out mutational signature detection from panel sequencing data. One example of its likelihood-based approach, SigMA, works surprisingly well even when the mutation count is extremely low. The simulated panel-based prediction of Signature 3-positive cases faithfully recapitulates the WGS-based results, and the drug response data provide experimental support. As thousands of cancer cases are being profiled by panels at many hospitals (Zehir et al., 2017) and more mutational signatures are characterized, the disclosed systems and methods will be fruitful in identifying the mechanisms underlying the mutations and whether they may be amenable to existing therapies.

For breast cancer, PARP inhibitors have been given only to BRCA1/2-mutant cases, but the disclosed results indicate that it may be expanded to a larger group of patients, depending on the exposure to Signature 3. Given that there are ˜270,000 newly diagnosed breast cancer cases in 2018 (Siegel et al., 2018), about 13,500-27,000 (5-10%) cases may be attributed to inherited mutations of BRCA1/2 (Roy et al., Nat Rev Cancer 2012). The disclosed analysis based on simulated data suggested that approximately twice that number of cases (27,000-54,000) may have HR defect (Signature 3) without inherited mutations, and the PARP inhibitors might be a promising option for these patients.

In ovarian cancer, PARP inhibitors have been used as a maintenance therapy after platinum-based chemotherapy, regardless of the BRCA1/2 mutation status (Ledermann et al., 2012). The general efficacy of PARP inhibitors in ovarian cancers regardless of the germline mutation status is in accordance with the widespread defect in the HR pathway in ovarian cancer, as reflected in the prevalence of Signature 3. In addition, other reports have suggested that ovarian cancers with the evidence of HR defect may exhibit a more favorable outcome to PARP inhibitors, compared to those without the evidence of HR defect (Mirza et al., 2016; Telli et al., 2016). This indicates that the genomic evidence of HR defect, including presence of Signature 3, could be a better predictive biomarker for PARP inhibitor response than the BRCA1/2 germline mutations. As shown in the outstanding efficacy of immune checkpoint blockades in microsatellite-unstable tumors of any origin tissues (Le et al., 2015), tumors with a common genome instability mechanism may share a selective vulnerability to treatments. It would be worthwhile to investigate whether the non-ovarian/breast cancers with Signature 3 could benefit from the PARP inhibitor treatments.

### Additional Explanations, Benefits, Methods, and Supplementary Information

Additional examples, explanations and benefits of the disclosed technologies are described in Gulhan et al., “Detecting the mutational signature of homologous recombination deficiency in clinical samples,” Nature Genetics, Apr. 15, 2019, the content of which is incorporated herein by reference in its entirety. Additionally, explanations and benefits of the disclosed technologies are described in the supplementary information of the foregoing article, in Gulhan et al., “Detecting the mutational signature of homologous recombination deficiency in clinical samples,” Nature Genetics, Supplementary Information, Apr. 15, 2019, the content of which is incorporated herein by reference in its entirety.

### Online Methods

Data availability: Whole-genome sequencing datasets from the TCGA project cohorts were downloaded from CGHub (http://cghub.ucsc.edu). The reads were aligned to the NCBI build 37 (hg19) using BWA-mem [ref]. Somatic mutation datasets from whole-genome sequencing of 80 additional breast tumor-normal pairs (Davies et al., 2017) were downloaded from http ://medgen.medschl . cam. ac.uk/serena-nik-zainal/.

SNV calls for tumors: the consensus SNV and indel call sets were utilized that werereleased by the Pan-Cancer Analysis of Whole Genomes (PCAWG) consortium. Consensus mutation calls for the MSK-IMPACT panel data (Zehir et al., 2017) were downloaded from cbioportal (http://cbioportal.org/msk-impact).

SNV calls and drug response data for cell lines: Mutation calls for the cancer cell lines from the Cancer Cell Line Encyclopedia (CLLE) were downloaded from (Basu et al., 2013) CCLE Data Portal (https://portals.broadinstitute.org/ccle/), and in vitro drug sensitivity information of relevant cancer cell lines to various compounds including PARP inhibitors were downloaded from Genomics of Drug Sensitivity in Cancer (GDSC; https://www.cancerrxgene.org/) (Yang et al., 2013).

### REFERENCES

Abkevich, V., Timms, K. M., Hennessy, B. T., Potter, J., Carey, M. S., Meyer, L. A., Smith-McCune, K., Broaddus, R., Lu, K. H., Chen, J., et al. (2012). Patterns of genomic loss of heterozygosity predict homologous recombination repair defects in epithelial ovarian cancer. Br. J. Cancer 107, 1776-1782.
Alexandrov, L., Kim, J., Haradhvala, N. J., Huang, M. N., Ng, A. W. T., Boot, A., Covington, K. R., Gordenin, D. A., Bergstrom, E., Lopez-Bigas, N., et al. (2018). The Repertoire of Mutational Signatures in Human Cancer. BioRxiv 322859.

Alexandrov, L. B., Nik-Zainal, S., Wedge, D. C., Campbell, P. J., and Stratton, M. R. (2013b). Deciphering Signatures of Mutational Processes Operative in Human Cancer. Cell Rep. 3,246-259.
Alexandrov, L. B., Nik-Zainal, S., Wedge, D. C., Aparicio, S. A. J. R., Behjati, S., Biankin, A. V., Bignell, G. R., Bolli, N., Borg, A., Børresen-Dale, A.-L., et al. (2013a). Signatures of mutational processes in human cancer. Nature 500, 415-421.
Alexandrov, L. B., Jones, P. H., Wedge, D. C., Sale, J. E., Campbell, P. J., Nik-Zainal, S., and Stratton, M. R. (2015). Clock-like mutational processes in human somatic cells. Nat. Genet. 47, 1402-1407.
Basu, A., Bodycombe, N. E., Cheah, J. H., Price, E. V., Liu, K., Schaefer, G. I., Ebright, R. Y., Stewart, M. L., Ito, D., Wang, S., et al. (2013). An interactive resource to identify cancer genetic and lineage dependencies targeted by small molecules. Cell 154, 1151-1161.
Blokzijl, F., Janssen, R., van Boxtel, R., and Cuppen, E. (2018). MutationalPatterns: comprehensive genome-wide analysis of mutational processes. Genome Med. 10.
Burns, M. B., Lackey, L., Carpenter, M. A., Rathore, A., Land, A. M., Leonard, B., Refsland, E. W., Kotandeniya, D., Tretyakova, N., Nikas, J. B., et al. (2013). APOBEC3B is an enzymatic source of mutation in breast cancer. Nature 494, 366-370.
Davies, H., Glodzik, D., Morganella, S., Yates, L. R., Staaf, J., Zou, X., Ramakrishna, M., Martin, S., Boyault, S., Sieuwerts, A. M., et al. (2017). HRDetect is a predictor of BRCA1 and BRCA2 deficiency based on mutational signatures. Nat. Med. 23, 517-525.
Drost, J., van Boxtel, R., Blokzijl, F., Mizutani, T., Sasaki, N., Sasselli, V., de Ligt, J., Behjati, S., Grolleman, J. E., van Wezel, T., et al. (2017). Use of CRISPR-modified human stem cell organoids to study the origin of mutational signatures in cancer. Science 358, 234-238.
Fedeles, B. I., Chawanthayatham, S., Croy, R. G., Wogan, G. N., and Essigmann, J. M. (2017). Early detection of the aflatoxin B1 mutational fingerprint: A diagnostic tool for liver cancer. Mol. Cell. Oncol. 4.
Frampton, G. M., Fichtenholtz, A., Otto, G. A., Wang, K., Downing, S. R., He, J., Schnall-Levin, M., White, J., Sanford, E. M., An, P., et al. (2013). Development and validation of a clinical cancer genomic profiling test based on massively parallel DNA sequencing. Nat. Biotechnol. 31, 1023-1031.
Fraser, M., Sabelnykova, V. Y., Yamaguchi, T. N., Heisler, L. E., Livingstone, J., Huang, V., Shiah, Y.-J., Yousif, F., Lin, X., Masella, A. P., et al. (2017). Genomic hallmarks of localized, non-indolent prostate cancer. Nature 541, 359-364.
Gehring, J. S., Fischer, B., Lawrence, M., and Huber, W. (2015). SomaticSignatures: inferring mutational signatures from single-nucleotide variants. Bioinforma. Oxf. Engl. 31, 3673-3675.
Gorthi, A., Romero, J. C., Loranc, E., Cao, L., Lawrence, L.A., Goodale, E., Iniguez, A. B., Bernard, X., Masamsetti, V. P., Roston, S., et al. (2018). EWS-FLI1 increases transcription to cause R-loops and block BRCA1 repair in Ewing sarcoma. Nature 555, 387-391.

Haradhvala, N. J., Kim, J., Maruvka, Y. E., Polak, P., Rosebrock, D., Livitz, D., Hess, J. M., Leshchiner, I., Kamburov, A., Mouw, K. W., et al. (2018). Distinct mutational signatures characterize concurrent loss of polymerase proofreading and mismatch repair. Nat. Commun. 9, 1746.
Huang, P.-J., Chiu, L.-Y., Lee, C.-C., Yeh, Y.-M., Huang, K.-Y., Chiu, C.-H., and Tang, P. (2018). mSignatureDB: a database for deciphering mutational signatures in human cancers. Nucleic Acids Res. 46, D964-D970.
Kasar, S., and Brown, J. R. (2016). Mutational landscape and underlying mutational processes in chronic lymphocytic leukemia. Mol. Cell. Oncol. 3.
Kazanov, M. D., Roberts, S. A., Polak, P., Stamatoyannopoulos, J., Klimczak, L. J., Gordenin, D. A., and Sunyaev, S. R. (2015). APOBEC-Induced Cancer Mutations Are Uniquely Enriched in Early-Replicating, Gene-Dense, and Active Chromatin Regions. Cell Rep. 13,1103-1109.
Lawson, C. L., and Hanson, R. J. (1995). Solving Least Squares Problems (Society for Industrial and Applied Mathematics).
Le, D. T., Uram, J. N., Wang, H., Bartlett, B. R., Kemberling, H., Eyring, A. D., Skora, A. D., Luber, B. S., Azad, N.S., Laheru, D., et al. (2015). PD-1 Blockade in Tumors with Mismatch-Repair Deficiency. N. Engl. J. Med. 372, 2509-2520.
Ledermann, J., Harter, P., Gourley, C., Friedlander, M., Vergote, I., Rustin, G., Scott, C., Meier, W., Shapira-Frommer, R., Safra, T., et al. (2012). Olaparib maintenance therapy in platinum-sensitive relapsed ovarian cancer. N. Engl. J. Med. 366, 1382-1392.
Lee, J., Lee, A. J., Lee, J.-K., Park, J., Kwon, Y., Park, S., Chun, H., Ju, Y. S., and Hong, D. (2018). Mutalisk: a web-based somatic MUTation AnaLyIS toolKit for genomic, transcriptional and epigenomic signatures. Nucleic Acids Res.
Meier, B., Volkova, N. V., Hong, Y., Schofield, P., Campbell, P. J., Gerstung, M., and Gartner, A. (2018). Mutational signatures of DNA mismatch repair deficiency in C. elegans and human cancers. Genome Res. 28, 666-675.
Mirza, M. R., Monk, B. J., Herrstedt, J., Oza, A. M., Mahner, S., Redondo, A., Fabbro, M., Ledermann, J. A., Lorusso, D., Vergote, I., et al. (2016). Niraparib Maintenance Therapy in Platinum-Sensitive, Recurrent Ovarian Cancer. N. Engl. J. Med. 375, 2154-2164.
Nik-Zainal, S., Alexandrov, L. B., Wedge, D. C., Van Loo, P., Greenman, C. D., Raine, K., Jones, D., Hinton, J., Marshall, J., Stebbings, L. A., et al. (2012). Mutational processes molding the genomes of 21 breast cancers. Cell 149, 979-993.
Nik-Zainal, S., Kucab, J. E., Morganella, S., Glodzik, D., Alexandrov, L. B., Arlt, V. M., Weninger, A., Hollstein, M., Stratton, M. R., and Phillips, D. H. (2015). The genome as a record of environmental exposure. Mutagenesis 30, 763-770.
Nik-Zainal, S., Davies, H., Staaf, J., Ramakrishna, M., Glodzik, D., Zou, X., Martincorena, I., Alexandrov, L. B., Martin, S., Wedge, D. C., et al. (2016). Landscape of somatic mutations in 560 breast cancer whole genome sequences. Nature 534, 47-54.
Northcott, P. A., Buchhalter, I., Morrissy, A. S., Hovestadt, V., Weischenfeldt, J., Ehrenberger, T., Grobner, S., Segura-Wang, M., Zichner, T., Rudneva, V. A., et al. (2017). The whole-genome landscape of medulloblastoma subtypes. Nature 547, 311-317.
Ohno, M., Sakumi, K., Fukumura, R., Furuichi, M., Iwasaki, Y., Hokama, M., Ikemura, T., Tsuzuki, T., Gondo, Y., and Nakabeppu, Y. (2014). 8-oxoguanine causes spontaneous de novo germline mutations in mice. Sci. Rep. 4, 4689.
Polak, P., Kim, J., Braunstein, L. Z., Karlic, R., Haradhavala, N. J., Tiao, G., Rosebrock, D., Livitz, D., Kübler, K., Mouw, K. W., et al. (2017). A mutational signature reveals alterations underlying deficient homologous recombination repair in breast cancer. Nat. Genet. 49, 1476-1486.
Roberts, K. G., Morin, R. D., Zhang, J., Hirst, M., Zhao, Y., Su, X., Chen, S.-C., Payne-Turner, D., Churchman, M. L., Harvey, R. C., et al. (2012). Genetic alterations activating kinase and cytokine receptor signaling in high-risk acute lymphoblastic leukemia. Cancer Cell 22, 153-166.
Rosales, R. A., Drummond, R. D., Valieris, R., Dias-Neto, E., and da Silva, I. T. (2017). signeR: an empirical Bayesian approach to mutational signature discovery. Bioinforma. Oxf. Engl. 33,8-16.
Rosenthal, R., McGranahan, N., Herrero, J., Taylor, B. S., and Swanton, C. (2016). deconstructSigs: delineating mutational processes in single tumors distinguishes DNA repair deficiencies and patterns of carcinoma evolution. Genome Biol. 17.
Sachs, N., de Ligt, J., Kopper, O., Gogola, E., Bounova, G., Weeber, F., Balgobind, A. V., Wind, K., Gracanin, A., Begthel, H., et al. (2018). A Living Biobank of Breast Cancer Organoids Captures Disease Heterogeneity. Cell 172, 373-386.e10.
Siegel, R. L., Miller, K. D., and Jemal, A. (2018). Cancer statistics, 2018. CA. Cancer J. Clin. 68, 7-30.
Telli, M. L., Timms, K. M., Reid, J., Hennessy, B., Mills, G. B., Jensen, K. C., Szallasi, Z., Barry, W. T., Winer, E. P., Tung, N. M., et al. (2016). Homologous Recombination Deficiency (HRD) Score Predicts Response to Platinum-Containing Neoadjuvant Chemotherapy in Patients with Triple-Negative Breast Cancer. Clin. Cancer Res. Off. J. Am. Assoc. Cancer Res. 22, 3764-3773.
Vilar Sanchez, E., Chow, A., Raskin, L., Iniesta, M. D., Mukherjee, B., and Gruber, S. B. (2009). Preclinical testing of the PARP inhibitor ABT-888 in microsatellite instable colorectal cancer. J. Clin. Oncol. 27, 11028-11028.
Waddell, N., Pajic, M., Patch, A.-M., Chang, D. K., Kassahn, K. S., Bailey, P., Johns, A. L., Miller, D., Nones, K., Quek, K., et al. (2015). Whole genomes redefine the mutational landscape of pancreatic cancer. Nature 518, 495-501.
Wu, Y.-M., Cieślik, M., Lonigro, R. J., Vats, P., Reimers, M. A., Cao, X., Ning, Y., Wang, L., Kunju, L. P., de Sarkar, N., et al. (2018). Inactivation of CDK12 Delineates a Distinct Immunogenic Class of Advanced Prostate Cancer. Cell 173, 1770-1782.e14.
Yang, W., Soares, J., Greninger, P., Edelman, E. J., Lightfoot, H., Forbes, S., Bindal, N., Beare, D., Smith, J. A., Thompson, I. R., et al. (2013). Genomics of Drug Sensitivity in Cancer (GDSC): a resource for therapeutic biomarker discovery in cancer cells. Nucleic Acids Res. 41, D955-D961.
Zamborszky, J., Szikriszt, B., Gervai, J. Z., Pipek, O., Póti, A., Krzystanek, M., Ribli, D., Szalai-Gindl, J. M., Csabai, I., Szallasi, Z., et al. (2017). Loss of BRCA1 or BRCA2 markedly increases the rate of base substitution mutagenesis and has distinct effects on genomic deletions. Oncogene 36, 746-755.
Zehir, A., Benayed, R., Shah, R. H., Syed, A., Middha, S., Kim, H. R., Srinivasan, P., Gao, J., Chakravarty, D., Devlin, S. M., et al. (2017). Mutational landscape of metastatic cancer revealed from prospective clinical sequencing of 10,000 patients. Nat. Med. 23, 703-713.
Zou, X., Owusu, M., Harris, R., Jackson, S. P., Loizou, J. I., and Nik-Zainal, S. (2018). Validating the concept of mutational signatures with isogenic cell models. Nat. Commun. 9, 1744.

1. A method of classifying a tumor of a patient, the method comprising:
obtaining a sample of a patient's tumor tissue;
performing DNA sequencing on the sample to output a set of genetic data;
performing a mutation analysis on the set of genetic data to output a set of mutations;
determining a sample mutational spectrum based on the set of mutations;
comparing the mutational spectrum to a set of clusters comprising different mutational spectrums to determine a matching cluster; and
outputting an indication of a mutational signature of the sample based on the matching cluster.

2. The method of claim 1, wherein comparing the set of clusters to determine a matching cluster further comprises:
performing a likelihood comparison to output a likelihood feature;
performing a cosine similarity measure to output a cosine similarity feature; and
inputting the likelihood feature and the cosine similarity feature into a gradient boosted machine trained for a specific tumor type using WSG data to output a matching score.

3. The method of claim 1, wherein performing DNA sequencing on the sample a mutation comprises performing DNA sequencing on a subset of the genes of the sample.

4. The method of claim 1, wherein outputting an indication further comprises outputting a recommended treatment for the patient based on the mutational signature.

5. The method of claim 1, wherein comparing comprises using a likelihood similarity measure.

6. The method of claim 1, wherein the tumor type comprises breast cancer, ovarian cancer, osteosarcoma, endometrial carcinoma, bladder cancer, medulloblastoma, prostate adenocarcinoma, Ewing's sarcoma, pancreatic adenocarcinoma, pancreatic neuroendocrine cancer, or esophageal adenocarcinoma.

7. The method of claim 1, wherein the set of genetic data comprises the whole genome of the sample.

8. The method of claim 2, wherein the subset of genes comprises between 50-20000 genes.

9. The method of claim 2, wherein the subset of genes comprises between 300-700 genes.

10. The method of claim 2, wherein the subset of genes comprises at least 300 genes.

11. The method of claim 2, wherein the subset of genes comprises 410 genes.

12. The method of claim 1, wherein the set of clusters are determined using WSG and based on which of 96 mutations are present in each sample.

13. The method of claim 1, wherein the set of clusters are determined using hierarchical clustering based on the fractional occurrence of each mutation in a sample.

14. The method of claim 1, wherein the mutational spectrums comprise probability distributions.

15. A method of classifying a tumor of a patient, the method comprising:
receiving a mutation analysis on a subset of genes on a sample of a patient's tumor tissue to output a set of mutations;
determining a sample mutational spectrum based on the set of mutations;
comparing the mutational spectrum to a set of clusters comprising different mutational spectrums to determine a matching cluster; and
determining a mutational signature of the sample based on the matching cluster; and
treating the patient based on the determined mutational signature.

16. The method of claim 15, wherein treating the patient comprises treating the patient with a PARP inhibitor or Po1 theta inhibitor if the mutational signature relates to homologous recombination deficiency.

17. The method of claim 15, wherein treating the patient comprises treating the patient with a treatment targeting homologous recombination deficiency if the mutational signature relates to homologous recombination deficiency.

18. The method of claim 15, wherein mutational signature relates to a deficiency in the DNA repair pathway.

19. A method of classifying a tumor of a patient, the method comprising:
receiving a gene analysis on a subset of genes on a sample of a patient's tumor tissue from to output a set of mutations;
determining a signature three mutation profile status based on the set of mutations; and
treating the patient with a PARP inhibitor based on the signature three mutation profile status.

20. The method of claim 19, wherein the tumor comprises breast cancer, ovarian cancer, osteosarcoma, endometrial carcinoma, bladder cancer, medulloblastoma, prostate adenocarcinoma, Ewing's sarcoma, pancreatic adenocarcinoma, pancreatic neuroendocrine cancer, or esophageal adenocarcinoma.
